SEQUENCE LISTING (52 sequences)

SEQ ID NO: 1 - DNA, 23 bp, Artificial Sequence
  Forward Primer Sequence Endogenous Oct3/4
tctttccaccaggcccccggctc

SEQ ID NO: 2 - DNA, 24 bp, Artificial Sequence
  Forward Primer Sequence Total Oct3/4
gtgagggccaggcaggagcacgag

SEQ ID NO: 3 - DNA, 23 bp, Artificial Sequence
  Forward Primer Sequence Endogenous Sox2
tagagctagactccgggcgatga

SEQ ID NO: 4 - DNA, 24 bp, Artificial Sequence
  Forward Primer Sequence Total Sox2
ggttacctcttcctcccactccag

SEQ ID NO: 5 - DNA, 24 bp, Artificial Sequence
  Forward Primer Sequence Endogenous Klf4
gcgaactcacacaggcgagaaacc

SEQ ID NO: 6 - DNA, 30 bp, Artificial Sequence
  Forward Primer Sequences Total Klf4
caccatggacccgggcgtggctgccagaaa

SEQ ID NO: 7 - DNA, 28 bp, Artificial Sequence
  Forward Primer Sequence Endogenous c-Myc
tgacctaactcgaggaggagctggaatc

SEQ ID NO: 8 - DNA, 24 bp, Artificial Sequence
  Forward Primer Sequence Total c-Myc
cagaggaggaacgagctgaagcgc

SEQ ID NO: 9 - DNA, 20 bp, Artificial Sequence
  Forward Primer Sequence Gapdh
gatgcccccatgtttgtgat

SEQ ID NO: 10 - DNA, 20 bp, Artificial Sequence
  Forward Primer Sequence Sox 17
ccatagcagagctcggggtc

SEQ ID NO: 11 - DNA, 18 bp, Artificial Sequence
  Forward Primer Sequence HNF3Beta  (Foxa2)
actggagcagctactacg

SEQ ID NO: 12 - DNA, 20 bp, Artificial Sequence
  Forward Primer Sequence Cytokeratin 19
ctgcagatgacttcagaacc

SEQ ID NO: 13 - DNA, 20 bp, Artificial Sequence
  Forward Primer Sequence Isl-1
gtttgtacgggatcaaatgc

SEQ ID NO: 14 - DNA, 18 bp, Artificial Sequence
  Forward Primer Sequence Nestin
ctaccaggagcgcgtggc

SEQ ID NO: 15 - DNA, 21 bp, Artificial Sequence
  Forward Primer Sequence Ngn3 (MATH4B)
tggcgcctcatcccttggatg

SEQ ID NO: 16 - DNA, 20 bp, Artificial Sequence
  Forward Primer Sequence Pax4
accagagcttgcactggact

SEQ ID NO: 17 - DNA, 19 bp, Artificial Sequence
  Forward Primer Sequence Pax6
tcacagcggagtgaatcag

SEQ ID NO: 18 - DNA, 20 bp, Artificial Sequence
  Forward Primer Sequence Pdx1 (IPF-1)
ctttcccgtggatgaaatcc

SEQ ID NO: 19 - DNA, 24 bp, Artificial Sequence
  Forward Primer Sequence Insulin 1
tagtgaccagctataatcagagac

SEQ ID NO: 20 - DNA, 21 bp, Artificial Sequence
  Forward Primer Sequence Glucagon
cattcacagggcacattcacc

SEQ ID NO: 21 - DNA, 21 bp, Artificial Sequence
  Forward Primer Sequence Amylase
caggcaatcctgcaggaacaa

SEQ ID NO: 22 - DNA, 21 bp, Artificial Sequence
  Forward Primer Sequence Glut-2
ttcggctatgacatcggtgtg

SEQ ID NO: 23 - DNA, 20 bp, Artificial Sequence
  Forward Primer Sequence IAPP
tgatattgctgcctcggacc

SEQ ID NO: 24 - DNA, 20 bp, Artificial Sequence
  Forward Primer Sequence PP
actagctcagcacacaggat

SEQ ID NO: 25 - DNA, 20 bp, Artificial Sequence
  Forward Primer Sequence Somatostatin
tcgctgctgcctgaggacct

SEQ ID NO: 26 - DNA, 20 bp, Artificial Sequence
  Forward Primer Sequence  Beta5-tubulin
tcactgtgcctgaacttacc

SEQ ID NO: 27 - DNA, 23 bp, Artificial Sequence
  Reverse primer sequence Endogenous Oct3/4
tgcgggcggacatggggagatcc

SEQ ID NO: 28 - DNA, 24 bp, Artificial Sequence
  Reverse primer sequence Total Oct3/4
ctgtagggagggcttcgggcactt

SEQ ID NO: 29 - DNA, 23 bp, Artificial Sequence
  Reverse primer sequence Endogenous Sox2
ttgccttaaacaagacgacgaaa

SEQ ID NO: 30 - DNA, 21 bp, Artificial Sequence
  Reverse Primer Sequence Total Sox2
tcacatgtgcgacaggggcag

SEQ ID NO: 31 - DNA, 23 bp, Artificial Sequence
  Reverse Primer Sequence Endogenous Klf4
tcgcttcctcttcctccgacaca

SEQ ID NO: 32 - DNA, 27 bp, Artificial Sequence
  Reverse Primer Sequence Total Klf4
ttaggctgttcttttccggggccacga

SEQ ID NO: 33 - DNA, 32 bp, Artificial Sequence
  Reverse Primer Sequence Endogenous c-Myc
aagtttgaggcagttaaaattatggctgaagc

SEQ ID NO: 34 - DNA, 28 bp, Artificial Sequence
  Reverse Primer Sequence Total c-Myc
ttatgcaccagagtttcgaagctgttcg

SEQ ID NO: 35 - DNA, 25 bp, Artificial Sequence
  Reverse Primer Sequence Gapdh
ttgctgacaatcttgagtgagttct

SEQ ID NO: 36 - DNA, 20 bp, Artificial Sequence
  Reverse Primer Sequence Sox 17
gtgcggagacatcagcggag

SEQ ID NO: 37 - DNA, 18 bp, Artificial Sequence
  Reverse Primer Sequence HNF3Beta  (Foxa2)
cccacataggatgacatg

SEQ ID NO: 38 - DNA, 20 bp, Artificial Sequence
  Reverse Primer Sequence Cytokeratin 19
ggccatgatctcatactgac

SEQ ID NO: 39 - DNA, 20 bp, Artificial Sequence
  Reverse Primer Sequence Isl-1
atgctgcgtttcttgtcctt

SEQ ID NO: 40 - DNA, 20 bp, Artificial Sequence
  Reverse Primer Sequence Nestin
tccacagccagctggacctt

SEQ ID NO: 41 - DNA, 20 bp, Artificial Sequence
  Reverse Primer Sequence Ngn3 (MATH4B)
agtcacccacttctgcttcg

SEQ ID NO: 42 - DNA, 20 bp, Artificial Sequence
  Reverse Primer Sequence Pax4
cccatttcagcttctcttgc

SEQ ID NO: 43 - DNA, 18 bp, Artificial Sequence
  Reverse Primer Sequence Pax6
cccaagcaaagatggaag

SEQ ID NO: 44 - DNA, 21 bp, Artificial Sequence
  Reverse Primer Sequence Pdx1 (IPF-1)
gtcaagttcaacatcactgcc

SEQ ID NO: 45 - DNA, 18 bp, Artificial Sequence
  Reverse Primer Sequence Insulin 1
cgccaaggtctgaaggtc

SEQ ID NO: 46 - DNA, 21 bp, Artificial Sequence
  Reverse Primer Sequence Glucagon
ccagcccaagcaatgaattcc

SEQ ID NO: 47 - DNA, 21 bp, Artificial Sequence
  Reverse Primer Sequence Amylase
cacttgcggataactgtgcca

SEQ ID NO: 48 - DNA, 21 bp, Artificial Sequence
  Reverse Primer Sequence Glut-2
agctgaggccagcaatctgac

SEQ ID NO: 49 - DNA, 20 bp, Artificial Sequence
  Reverse Primer Sequence IAPP
ggaggactggaccaaggttg

SEQ ID NO: 50 - DNA, 20 bp, Artificial Sequence
  Reverse Primer Sequence PP
agacaagagaggctgcaagt

SEQ ID NO: 51 - DNA, 23 bp, Artificial Sequence
  Reverse Primer Sequence Somatostatin
gccaagaagtacttggccagttc

SEQ ID NO: 52 - DNA, 20 bp, Artificial Sequence
  Reverse Primer Sequence  Beta5-tubulin
ggaacatagccgtaaactgc

(19) United States
(12) Patent Application Publication
(10) Pub. No.: US 2015/0342999 A1
(43) Pub. Date: Dec. 3, 2015

(54) METHODS AND COMPOSITIONS FOR TREATING DIABETES WITH IPS DERIVED PANCREATIC BETA-LIKE CELLS

(71) Applicants: Yupo Ma, Setauket, NY (US); Milton Waner, New York, NY (US)

(72) Inventors: Yupo Ma, Setauket, NY (US); Milton Waner, New York, NY (US)

(21) Appl. No.: 13/582,963

(22) PCT Filed: Mar. 7, 2011
(86) PCT No.: PCT/US11/27452

§ 371 Date: Dec. 18, 2012

Related U.S. Application Data
(60) Provisional application No. 61/339,510, filed on Mar. 5, 2010.

(51) Int. Cl. A61K 35/545; C12N 5/074

(52) U.S. Cl. CPC A61K 35/545; C12N 5/0696

(57) ABSTRACT

Diabetes mellitus is characterized by either the inability to produce insulin (Type 1 diabetes) and or as insensitivity to insulin secreted by the body (Type 2 diabetes). In either case, the body is unable to efficiently move blood glucose across cell membranes to be utilized. This leads to a variety of local and systemic detrimental effects. Current treatments for diabetes focus on exogenous insulin administration and dietary control. Provided herein are treatments of diabetes using a cellular therapy to ameliorate symptoms associated with both reduced insulin secretion and insulin sensitivity. Using induced-pluripotent stem (iPS) cells, beta-like (β-like) cells similar to the endogenous insulin secreting cells were derived. These β-like cells secreted insulin in response to glucose, and corrected a hyperglycemic phenotype in a mouse model of Type 2 diabetes via an iPS cell transplant. Within the Type 2 diabetes mouse model, a long term correction of hyperglycemia was achieved as measured by blood glucose and hemoglobin Alc measurements. Reduction of hyperglycemia was also seen in a chemically-induced mouse model for Type 1 diabetes.

### CROSS REFERENCE TO RELATED. APPLICATIONS

This application is related is U.S. provisional application 61/339,510, filed Mar. 5, 2010.

### FIELD OF THE INVENTION

The present invention relates generally to stems cells, including induced pluripotent stem cells (iPS) and their use to treat diseases, such as diabetes.

### BACKGROUND

Approximately 3.5% of the US population is diagnosed with diabetes mellitus, which makes this is a major health care problem. Type 1 diabetes mellitus is an autoimmune disease where insulin-secreting β-cells in pancreatic islets are irreversibly destroyed by autoimmune assault, resulting in a lack of insulin production. Type 2 diabetes mellitus occurs when the pancreas produces insufficient amounts of the hormone insulin and/or the body's tissues become resistant to normal or even high levels of insulin. In either case, introduction of exogenous insulin is usually enough to ameliorate the symptoms. However, insulin dosage is difficult to adjust. Furthermore, exogenous insulin frequently fails to achieve optimal glucose control even when intensive regimens, such as multiple daily insulin pump infusions with frequent monitoring of blood glucose, are used. These often lead to an increased incidence and severity of hypoglycemic episodes. Cell-based therapies are intended to reduce the dependence of diabetic patients on insulin injections via the introduction of exogenous insulin secreting cells, resulting in endogenous insulin production. One promising avenue by which this can occur is through the use of stem cells (1-7).

Current cell therapies for diabetes focus primarily on either whole pancreas transplant, or by introducing pancreatic islets into the portal vein of a diabetic individual (8). While these techniques are largely effective, they are hindered by immune rejection and the lack of primary tissues for transplantation. The concept of autologous grafting of insulin-secreting cells derived from the patient's own tissue stem cells, particularly derived from bone marrow (BM) or liver, is attractive (3). However, transplantation of these cells in animals encounters problems with restricted growth capacity, low levels of insulin expression and poor, or non-existent, insulin secretion. More recent studies suggest that BM stem cells improve experimental diabetes in viva by enhancing the regeneration and survival of existing (cells rather than repopulating the islets with newly trans-differentiated β-cells (2, 9).

Recently, methods to retrodifferentiate adult somatic cells into induced pluripotent stem (iPS) cells using defined transcription factors have been established (10-13). These iPS cells, to date, are quite similar to embryonic stem cells and have the same pluripotent characteristics. Because iPS cells can potentially have the same haplotype as the host, immune rejection may be avoided. Specific cell types derived from iPS cells have been used to treat various diseases in mouse models, including sickle cell anemia (14), Parkinson's disease (15) and hemophilia A (16). In view of the foregoing, it would be desirable to provide iPS cells that can be used in methods to treat diabetes.

One barrier to more widespread use of stem cells, including iPS cells, therapeutically is the risk of tumor formation and the inability to control stem cell fate when they are introduced into the body. If transplanted cells do not integrate properly, they may retain the ability to differentiate and expand, increasing the risk of inappropriate development, leading to deformation or even teratoma or cancer formation. Therefore, it would be desirable to provide stem cells whose fate can be controlled after they are introduced into the body and methods for generating such stem cells.

### SUMMARY OF THE INVENTION

Applicants demonstrate, inter alia, that iPS cells derived from adult somatic cells, such as skin fibroblasts, can differentiate into insulin secreting β-like cells in vitro and that they respond to glucose stimulation under physiologic or pathologic conditions. The resulting insulin secreting β-like cells were introduced via intraportal vein injection into the livers of two different mouse systems, modeling Type 1 and Type 2 diabetes. The β-like cells engrafted and corrected the hyperglycemic phenotype in both models demonstrating that the reprogrammed somatic cells can be used to treat diabetes Type 1 or 2.

In one aspect, cells are provided where the cells are induced pluripotent stem cells (iPS) that secrete insulin. In some embodiments, the cells are human cells. In some embodiments, the iPS cells are derived from fibroblasts. In some embodiments, the iPS cells are derived from adult somatic cells wherein generation of multi-lineage progenitors is performed without trypsinization (See FIG. 1, Stage 2). In some embodiments, the iPS cells can secrete insulin for at least about 20 days, at least about 30 days, at least about 8 weeks, at least about 3 months, at least about six months, at least about nine months, at least about one year or at least about two years.

In another aspect, methods of treating diabetes are provided comprising administering to a diabetic subject induced pluripotent stem cells (iPS) that secrete insulin. In some embodiments, the diabetic subject is human. In some embodiments, the diabetic subject has Type 1 diabetes. In some embodiments, the diabetic subject has Type 2 diabetes. In some embodiments, the diabetic subject is still responsive to insulin. In some embodiments, the diabetic subject is resistant to insulin. In some embodiments, the iPS cells administered are autologous (i.e., derived from the diabetic subject). In some embodiments, the iPS cells are administered by engrafting to liver parenchyma via intraportal vein injection. In some embodiments, at least about 200,000 iPS cells are administered. In some embodiments, the iPS cells are derived from adult somatic cells wherein generation of multi-lineage progenitors is performed without trypsinization (See FIG. 1, Stage 2). In some embodiments, the iPS cells can secrete insulin for at least about 20 days, at least about 30 days, at least about 8 weeks, at least about 3 months, at least about six months, at least about nine months, at least about one year or at least about two years.

In yet another aspect, methods of generating induced pluripotent stem cells (iPS), as well as the iPS cells produced by these methods, are provided that include transforming adult somatic cells with a vector that transiently expresses transcription factors that induce pluripotency. In some embodiments, the adult somatic cell is a human cell. In some embodiments, the adult somatic cell is a fibroblast. In some embodiments, the vector can not replicate in the transformed cell. In some embodiments, the vector can not integrate into the cell's chromosome. In some embodiments, the vector is derived from a baculovirus. In some embodiments, the transcription factors are selected from Oct4, Lin28, Nanog and/or Sox2.

In yet a further aspect, methods of generating stem cells (iPS), as well as the stem cells produced by these methods, are provided that include integrating a heterologous DNA into the chromosome of the cell by homologous recombination, thereby generating an inducible suicide gene in the cell. In some embodiments, the stem cells are human stem cells. In some embodiments, the stem cells are induced pluripotent stem cells. In some embodiments, the heterologous DNA includes an inducible promoter (e.g., an inducible promoter turned on by tetracycline or a tetracycline derivative). In some embodiments, the heterologous DNA includes a drug selection cassette. In some embodiments, the suicide gene is selected from caspase-9, caspase-8, caspase-2, BH3 interacting domain death agonist (BID) or Herpes Simplex Virus-1 thymidine kinase.

It is contemplated that whenever appropriate, any embodiment of the present invention can be combined with one or more other embodiments of the present invention, even though the embodiments are described under different aspects of the present invention.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

The above description sets forth rather broadly the more important features of the present invention in order that the detailed description thereof that follows may be understood, and in order that the present contributions to the art may be better appreciated. Other features and advantages of the invention will be apparent from and encompassed by the following figures, detailed description and claims.

### BRIEF DESCRIPTION OF THE DRAWINGS

FIG. 1 shows a time line for the selective differentiation of iPS cells into insulin-producing β-like cells. The schematic representation of the 3-stage differentiation protocol for deriving insulin-secreting β-like cells from ES-like iPS cells is as follows—Stage 1: iPS cells differentiate into embryo-like spheroids, Stage 2: generation of multi-lineage progenitors which migrate from the embryoid bodies, and Stage 3: selective differentiation of iPS cells into insulin-secreting β-like cells.

FIG. 2 depicts characterizations of iPS cell derived insulin producing β-like cells. FIG. 2A shows immunofluorescence staining of the cells at Stage 3, Day 20. In vitro derived β-like cells stained with antibodies to GFP, nestin and insulin at day 13 of stage 3. Cells counterstained with DAPI. Nestin staining is reduced to minimal, while insulin staining persisted. FIG. 2B shows gene expression profile of in vitro derived β-like Cells. Gene expression analysis was done by RT-PCR with various pancreatic cell markers during iPS cells-derived β-like cell differentiation. Total RNA was isolated from cells at stage 1 (S1), stage 2 (S2) and stage 3 (S3) of β-like cell development. B5-tubulin was used as endogenous control. iPSC=cells before differentiation.

FIG. 3 shows in vitro insulin induction. Upon glucose treatment, stage 3, day 20 iPS derived β-like cells secrete insulin in vitro. Cells were first exposed to 5 glucose concentrations (5 mM, 10 mM, 20 mM, 30 mM and 40 mM) and the insulin levels in their supernatants were determined. Glucose was then removed from the medium and insulin levels were again measured in supernatants from the same cells.

FIG. 4 shows the in vivo characterization of a diabetes mellitus Type 2 model. FIG. 4A shows the glucose concentration in an untransplanted Type 2 diabetes mellitus mouse model. Basal glucose concentrations obtained from untransplanted Type 2 diabetes mellitus mouse models (n=20). As shown, this model began to exhibit diabetic features (glucose>300 mg/dl) at 3 weeks of age. Glucose levels continue to increase until week 6, and then remain elevated at 600 mg/dl. Normal control animals retain lower glucose levels throughout the entire time period. FIG. 4B shows insulin levels in an untransplanted Type 2 diabetes mellitus mouse model. Basal insulin concentrations obtained from untransplanted Type 2 diabetes mellitus mouse models at 4 weeks old n=20), 8 weeks old (n=3), 12 weeks old (n=3) and 16 weeks old (n=3). Insulin is initially high at 4 weeks, but drops off and remains reduced from 8 weeks on. Insulin concentrations from normal mice (n=3) are shown for comparison. FIG. 4C shows the results of an insulin tolerance test in an untransplanted Type 2 diabetes mellitus mouse model. The models were injected intraperitoneally (i.p.) with 0.75 U/kg human insulin at 4 weeks old (n=3), 8 weeks old (n=3), and 16 weeks old (n=3). Fasting glucose concentration was measured prior to injection and at 30 min, 60 min and 90 min post injection via tail vein bleed and hand held glucometer. At 4 weeks, the mice show sensitivity to insulin therapy, but were resistant by 8 and 16 weeks of age.

FIG. 5 shows the in vivo characterization of a transplanted diabetes mellitus Type 2 model. FIG. 5A shows the glucose concentration in an engafted Type 2 diabetes mellitus mouse model. Prior to transplantation, all mice present a hyperglycemic phenotype. Stage 3, day 7 iPS cell derived pancreatic beta cells engrafted into Type 2 diabetes mellitus mice (n=30) were able to regulate glucose levels and ameliorate hyperglycemia for more than 2 months. Glucose readings were obtained from tail vein and measured by a hand held glucometer every 2-3 days. For untreated and control mice, n is as marked. See FIG. 15 for weekly tracking of the decrease in treated mice due to both post-surgical loss and harvest of animals for histology. FIG. 5B shows insulin production in an engrafted Type 2 diabetes mellitus mouse model. Serum insulin levels were measured 3 weeks posttransplantation in Type 2 diabetes mellitus mice (n=30) engrafted with stage 3 day 7 iPS cell derived β-like cells. The data indicate that treated mice had markedly increased insulin levels as compared to untreated mice. FIG. 5C show Hemoglobin Alc measurements in an engrafted Type 2 diabetes mellitus mouse model. At 4 weeks post-transplantation, blood samples from engrafted Type 2 diabetes mellitus mice (n=3) were tested by an independent company (DTI) to measure the level of Hemoglobin Alc. The Hb Alc level of unengrafted mice is approximately 2.5 times higher than normal. After engraftment, Hb Alc in the treated mice (n=3), while still higher than normal control mice, has decreased to a level approximately 40% lower than the untreated cohort (n=3).

FIG. 6 shows immunohistochemistry and immunofluorescence stained liver tissue from an engrafted Type 2 diabetes mellitus mouse model. Mice were sacrificed at 7 days (n=3 for each) for immuno-staining of liver tissues to analyze distribution of engrafted cells. Panel A shows liver of C57BL6 control as negative control for GFP staining, Panel B shows, at low magnification, the engraftment of spindle-shaped GFP positive cells (brown) scattered throughout the liver parenchyma, Panel C shows higher magnification of the GFP positive cells. At high magnification on panel D, GFP positive cells (green) are scattered throughout the tissue. Panel E shows the same section with insulin positive cells (red), and panel F shows the merged images to demonstrate co-localization of GFP and insulin.

FIG. 7 shows glucose levels in a Type 1 diabetes mellitus mouse model. The Type 1 diabetes mellitus mouse model was derived from a single i.p. injection of 180 mg/kg Streptozotocin and stabilized for 10 days prior to transplantation. The mice used in this study (n=5) all demonstrated hyperglycemia of greater than 400 mg/dl glucose in at least 3 readings prior to transplantation. Two mice were then injected with iPS cell derived β-like cells via hepatic portal vein injection. Fasting glucose readings obtained every 2-3 days starting from two days post transplantation show amelioration of hyperglycemia.

FIG. 8 shows morphologies of parental skin fibroblasts expressing Green Fluorescence Protein (GFP) and iPS cells (2 subclones) after somatic cell reprogramming. At 20 days post induction, low magnification phase contrast images show two subclones, iPSC SC1 and iPSC SC2, that are distinct from parental skin fibroblasts. GFP expression was still highly expressed after reprogramming.

FIG. 9 shows quantitative RT-PCR analysis of Oct4, Sox2, Klf4 and c-Myc in two iPS cell lines. The expression patterns of two iPS cell subclones, iPSC SC1 and iPSC SC2, demonstrate ESC-like characteristics. W4 ES cells (mouse ES cells) were used as the positive control and normal fibroblasts were used as the negative control. Housekeeping gene, GAPDH, was used as an endogenous control. Primers were used that will only amplify endogenous genes of Oct4, Sox2, Klf4 and cMyc, labeled “Endog” on figure. Also tested was Whether retroviral transcripts were silenced in newly established iPS cells clones by amplifying the genes of the endogenous and total expressions(25). Clones with silenced retroviral transcripts will have indistinguishable “Endog” and “Total” gene expressions.

FIG. 10 shows iPS cells characterized by immunofluorescence staining using well established pluripotency markers: Oct3/4, Sox2, Nanog and SSEA-1. The clones were counterstained with DAPI (blue), confirming the expression of ES cell markers throughout the iPS cell clones.

FIG. 11 depicts histological analyses of teratoma. Low (A, B, C) and High (D, E, F) magnification images confirm the presence of cell lineages belonging to the three germ layers, ectoderm (skin), mesoderm (cartilage) and endoderm (gut-like) within the tumor sections.

FIG. 12 shows morphologies of differentiating iPS cells into β-like cells. Images of iPS cells at 3 different time points during stage 3 differentiation into β-like□ cells in vitro. Green fluorescence protein continues to be expressed throughout all stages.

FIG. 13 shows immunofluorescence staining: Stage 3, Day 7. In vitro derived β-like cells stained with antibodies to GFP, nestin and insulin at day 7 of stage 3. Cells counterstained with DAPI.

FIG. 14 shows immunofluorescence staining: Stage 3, Day 13. In vitro derived β-like cells stained with antibodies to GFP, nestin and insulin at day 13 of stage 3. Cells counterstained with DAPI. Both nestin and insulin continue to stain strongly.

FIG. 15 plots the number of animals surviving following surgery by week. The number of treated Type 2 animals surviving at each week of the study. Post transplant losses were determined to be due to post surgical complications, as described below.

FIG. 16 shows insulin resistance tests for two relapsed mice. Two out of the 30 transplanted mice relapsed into a diabetic hyperglycemic phenotype 3-4 weeks post engraftment. They were subjected to the glucose tolerance test described below. Glucose readings obtained at 30, 60, 90 and 120 min post injection demonstrated that both mice had become resistant to insulin.

FIG. 17 characterization of three factor iPS (3F-iPS) cells. Immunofluorescent assays of 3F-iPS colonies using pluripotency markers SALL4, Nanog, SSEA-1, and Oct4. All 3F-iPS colonies were positive for all 4 markers.

FIG. 18 shows western blots depicting expression of Lin 28, Oct4, Nanog, Sox2. Lane 1, embryonic stein (ES), lane 2, Fibroblasts infected with BacMan viruses expressing 4 defined transcription factors. Lane 3, control fibroblasts.

FIG. 19 depicts the hypothesized mechanisms of transplanted cells in the prevention of insulin resistance and beta cell failure with reference to the Type 2 diabetes mouse models.

FIG. 20 depicts schematic representations of homologous recombination. (A) Homologous recombination via “crossing-over” during meiosis in eukaryotes. (B) Schematic mechanism of homologous recombination.

FIG. 21 depicts a cassette for introducing a gene of interest into a genomic sequence according to some embodiments of the present invention.

### DETAILED DESCRIPTION OF THE INVENTION

The ability of iPS cells to differentiate in vitro into insulin-secreting cells is described herein, and these cells are further shown to be functional in vivo to correct a hyperglycemic phenotype in diabetic mouse models. The iPS cells described herein have a number of advantages including, but not limited to 1) the iPS cells were derived from fibroblasts of C57BL6 background mice, close relatives of the diabetic mouse models used here, and thus reduce the possibility of graft rejection upon transplantation, 2) insulin produced from the transplanted iPS cell derived β-like cells can be considered endogenous insulin, thereby allowing maintenance of normal glucose homeostasis as the mice age, and 3) an unlimited supply of cells for differentiation can be produced from the iPS cells.

A “stem-cell” is a cell characterized by the ability of self-renewal through mitotic cell division and the potential to differentiate into a tissue or an organ.

The term “subject” refers to any individual, human or animal, that belongs to a species that normally produces insulin.

An “induced pluripotent stem (iPS) cell” is any type of cell derived from a non-pluripotent cell, which can include, but is not limited to, a hematopoietic cell, a mesenchymal cell, an epithelial cell, a skin cell, a neural cell, or any type of non-pluripotent somatic stem cell.

The term “pluripotent” or “pluripotency” refers to cells with the ability to give rise to progency that can undergo differentiation, under appropriate conditions, into cell types that collectively exhibit characteristics associated with cell lineages from endoderm, mesoderm, and ectoderm. iPS cells can contribute to tissues of a prenatal, postnatal, or adult organism.

The term “somatic cell” refers to a cell that is not a germline cell. An example of a somatic cell is a fibroblast cell, although one skilled in the art would appreciate that a multitude of different types of somatic cells exist.

The term “β-like cell” refers to a cell derived from an iPS cell that has the same characteristics as a naturally occurring β cell and is an insulin secreting cell. One of skill in the art would recognize that an iPS cell has the potential to differentiate into a β-like cells, and the β-like cells as described herein are cells that have been differentiated from the iPS cells of the present disclosure and have the ability to secrete insulin.

An “insulin-secreting cell” is any type of cell that inter alma, secretes insulin.

The term “autologous iPS” refers to iPS cells induced from cells derived from the subject into which the iPS cells will be reintroduced.

The term “reprogramming” refers to the process of dedifferentiating a non-pluripotent cell into a cell exhibiting pluripotent stem cell characteristics.

The term “vector” refers to any type of genetic element, such as a plasmid, phage, transposon, cosmid, chromosome, virus, etc., that is capable of replicating when provided with appropriate control and accessory elements and which can transfer exogenous gene sequences into cells, including all manner of cloning and expression vehicles, as well as viral vectors. A cell is “transformed” following introduction of said vector.

The term “homologous recombination” refers to the exchange of DNA fragments between two DNA molecules (during crossover). The fragments that are exchanged are flanked by sites of identical nucleotide sequences between the two DNA molecules (i.e., homology DNA regions).

The term “integrate” or “chromosomal integration” refers to the process in which a DNA segment is introduced into a cell and becomes congruent with the chromosome through recombination between homologous DNA regions on the introduced DNA segment and within the chromosome.

The term “heterologous DNA” refers to DNA that is not endogenous to the cell into which it is introduced, but can include an endogenous DNA sequence containing modification.

The term “suicide gene” refers to a gene encoding a protein whose expression results in the death of cells expressing the gene.

The term “inducible promoter” refers to a polynucleotide sequence that initiates and facilitates the transcription of a gene when the host cell comprising the inducible promoter is exposed to some particular external stimulus. The activity of an inducible promoter may be triggered, for example, by chemical, physical, nutritional or cell growth factors.

The term “engrafting” or “engraftment” or “engraft” refers to the process of infused or transplanted donor stem cells to the host organism. As used herein, stem cells can engraft anywhere in the host organism.

The term “treating” or “treat” or “treatment” refers to the relief or alleviation of at least one symptom of a disorder in a subject.

The term “administer” or “administering” refers to the dispensing, supplying, applying, giving, apportioning, or contributing stem cells to a subject in need thereof. Routes for administration can include, but are not limited to, oral, subcutaneous or parenteral, including intravenous, intraportal, intraarterial, intramuscular, intraperitoneal, intranasal, as well as by infusion.

The term “multi-lineage progenitors” refers to stem cells or progenitor cells capable of differentiating to mesoderm, ectoderm and/or endoderm lineages.

The term “transcription factor” refers to polynucleotides and/or polypeptides that are involved in gene regulation in both prokaryotic and eukaryotic organisms.

During in vitro differentiation of the iPS cells to β-like cells, the Applicants determined that a refinement of the differentiation protocol (17) significantly improved the efficiency of the differentiation process. By eliminating a trypsinization step between stages 2 and 3, a substantial increase of 80% in the survival of the differentiating cells was seen, resulting in a more robust cell yield.

The use of two different mouse models demonstrated that the iPS cells can be used for treating both Type 1 or Type 2 diabetes via the cellular transplant route. In Type 2 model mice, the mice maintained a hyperglycemic phenotype and had insulin production that was rapidly depleted as the mice aged. Three key features are hallmarks of Type 2 diabetes: hyperglycemia, progressive failure of insulin secretion and the development of insulin resistance. Impairment in insulin secretion is likely caused by β-cell exhaustion due to a constant, unsuccessful attempt to compensate for the existing insulin resistance. In addition, β-cell function is adversely affected by glucotoxicity, generating a downward cycle of hyperglycemia leading to decreased insulin secretion, which further worsens hyperglycemia.

Transplantation of these animals with one dose containing as few as 200,000 iPS derived β-like cells showed that the cells engrafted well and persistently in the liver and resulted in restored insulin secretion and normalization of glucose levels within two days post implantation. The injection of 1×10⁶ cells provided no detectable improvement in serum insulin levels above the response Obtained with the lower cell dose. The control of glycemia was maintained for approximately 20-30% of the life expectency of these mice. No significant weight reduction was observed in the iPS-derived cell transplanted mice as compared to the control mice, Lepr^{db} (data not shown).

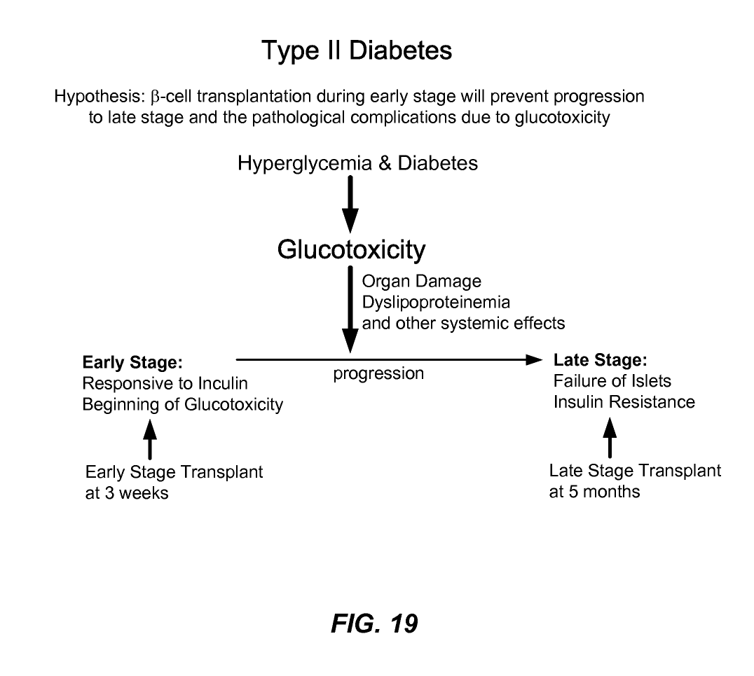

Without wishing to be bound by theory, in Type 2 diabetes, impairment in insulin secretion is likely caused by β-cell exhaustion due to a constant, unsuccessful attempt to compensate for the existing insulin resistance. In addition, β-cell function is adversely affected by glucotoxicity, generating a downward cycle of hyperglycemia leading to decreased insulin secretion, which further worsens hyperglycemia. Applicants hypothesize that transplanted insulin producing iPS cells are able to compensate for the progressive failure of endogenous islet cells by secreting insulin, thus controlling blood glucose levels so that endogenous β-cell damage and destruction by glucotoxicity can be avoided and the islet cells in the pancreas can replicate (FIG. 19). Therefore, the progression of Type 2 diabetes begins a loss of insulin secretion inducing a hyperglycemic condition with concomitant glucotoxicity. Insulin resistance follows shortly thereafter, and feeds back into the worsening condition as described above. Therefore, in some embodiments, insulin producing iPS cells will be transplanted at an early stage (3 weeks in the case of mice) of the disease. While in other embodiments, insulin producing iPS cells will be transplanted later in the disease progression (5 months in the case of mice) after resistance is well entrenched.

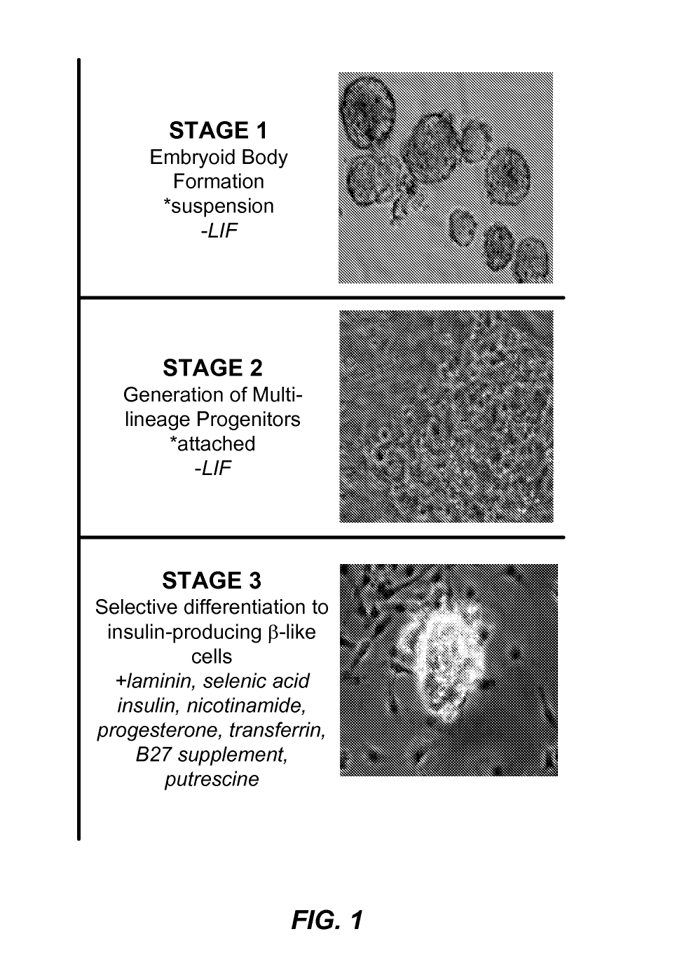

In one aspect, cells are provided where the cells are induced pluripotent stem cells (iPS) and secrete insulin. In some embodiments, the cells are human cells. In some embodiments, the iPS cells are derived from fibroblasts. In some embodiments, the iPS cells are derived from adult somatic cells wherein generation of multi-lineage progenitors is performed without trypsinization (See FIG. 1. Stage 2). In some embodiments, the iPS cells can secrete insulin for at least about 20 days, at least about 30 days, at least about 8 weeks, at least about 3 months, at least about six months, at least about nine months, at least about one year or at least about two years.

In another aspect, the iPS cells can include microcarriers comprising a matrix being of a size to permit the aggregation and attachment of the iPS cells to the matrix to assist in the engrafting of the iPS cells. Such matrices are known in the art, and can include but are limited to, charged and uncharged particles and can include any additional extracellular matrix components known in the art capable of supporting growth of stein cells. Examples of components known in the art capable of supporting growth of stem cells can include, but are not limited to, polysaccharides, proteins, protoglycans, glycoproteins, glycosaminoglycans, fibrous proteins such as elastin, fibronectin, laminin, and collagen.

In another aspect, methods of treating diabetes are provided comprising administering to a diabetic subject induced pluripotent stem cells (iPS) that secrete insulin. In some embodiments, the diabetic subject is human. In some embodiments, the diabetic subject has Type 1 diabetes. In some embodiments, the diabetic subject has Type 2 diabetes. In some embodiments, the diabetic subject is still responsive to insulin. In some embodiments, the diabetic subject is resistant to insulin. In some embodiments, the iPS cells administered are autologous (i.e., derived from the diabetic subject). In some embodiments, at least about 200,000 iPS cells are administered. In some embodiments, the iPS cells are derived from adult somatic cells wherein generation of multi-lineage progenitors is performed without trypsinization (See FIG. 1, Stage 2). In some embodiments, the iPS cells can secrete insulin for at least about 20 days, at least about 30 days, at least about 8 weeks, at least about 3 months, at least about six months, at least about nine months, at least about one year or at least about two years. In another embodiment, the iPS cells are α-cells that synthesize and secrete glucagon. In some embodiments, the iPS cells administered to a subject are a mixture of u-cells and β-cells.

In some embodiments, the iPS cells are administered by engrafting to liver parenchyma via intraportal vein injection. One of skill in the art will recognize that insulin producing iPS cells can be administered, e.g., by injection, at other sites. For example, insulin producing iPS cells can be injected into the testicles or sub-renal capsule.

In yet another aspect, methods of generating induced pluripotent stem cells (iPS), as well as the iPS cells produced by these methods, are provided that include transforming adult somatic cells with a vector that transiently expresses transcription factors that induce pluripotency. In some embodiments, the adult somatic cell is a human cell. In some embodiments, the adult somatic cell is a fibroblast. In some embodiments, the vector can not replicate in the transformed cell. In some embodiments, the vector can not integrate into the cell's chromosome. In some embodiments, the vector is derived from a baculovirus. In some embodiments, the transcription factors are selected from Oct4, Lin28, Nanog and/or Sox2. While not limited to, examples of said transcription factors can be found expressed as GenBank accession numbers ABF29403.1 or ADW77327.1 (Oct4), AAH28566.1 (Lin28), AAP49529.1 (Nanog) and NP_{—}003097.1 (Sox2). One skilled in the art would appreciate that polynucleotides encoding said transcription factors can vary depending on the degenerate nature of the genetic code. One skilled in the art would further appreciate that homologs of said transcription factors having a percent identity, for example, 99%, 98%, 97%, 96%, 95%, 90%, 85%, 80%, 75%, 70%, can served as functional equivalents in the generation of iPS cells.

Percent identity can be determined by aligning two sequences to be compared, determining the number of identical residues in the aligned portion, dividing that number by the total number of residues in the sequence that provides the basis for comparison, and by multiplying the result by 100. A percentage identity can also be determined with reference to a specified region of a polypeptide against another polypeptide or region thereof. To determine percent identity, sequences can be aligned using methods and computer programs identifiable by a skilled person. “Sequence alignment” indicates the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA [9, 10]. When using all of these programs, the preferred settings are those that results in the highest sequence similarity.

iPS cells as described herein can be generated in any way as known in the art, which can include, but is not limited to, transfection of exogenous stem cell-inducible genes and/or the channeling of the protein products of such genes into a non-pluripotent stem cell.

Introduction of Suicide Genes into Stem Cells to Control the Stem Cell Fate

While stem cells, including iPS cells, could be used to treat of a variety of diseases, including diabetes, or tissue repair, they carry oncogenic risks. Therefore, it would be desirable to be able to control stem cell fate after they are introduced into the body. A fate-controllable stem cell as described herein refers to a stem cell whose fate can be controlled after transplantation of the stem cell after it is introduced into the body. In one aspect, embodiments of the invention relate generally to introduction of suicide genes into stem cells using a homologous recombination.

In some embodiments, heterologous DNA is integrated into the chromosome of the stem cell using homologous recombination to control stem cell fate after transplantation into the body. The heterologous DNA can include, but is not limited to, promoter regions (inducible) that is integrated upstream of endogenous genes, coding sequences for one or more genes, drug selection cassettes, or the introduction of entire expression cassettes of suicide genes that include promoter regions and coding sequences for one or more genes. The concept of suicide genes is well known in the art. The suicide genes can be chosen from endogenous pro-apoptotic genes, for example, caspase-9, caspase-8, caspase-2, BH3 interacting domain death agonist (BID) or heterologous genes, for example, herpes simplex virus-1 thymidine kinase (HSV-1TK). One skilled in the art would understand that a variety of suicide genes can be used to control the fate of the stem cells after transplantation. The suicide genes can be controlled by any inducible promoter as is known in the art, including, but not limited to, the Tet-On (Clonetech, Mountain View, Calif.), which is induced by the tetracycline derivative doxycycline, or by a homodimerization system. The induced suicide genes can be introduced into any sites on the chromosome, but non-functional regions in the chromosomes are preferred.

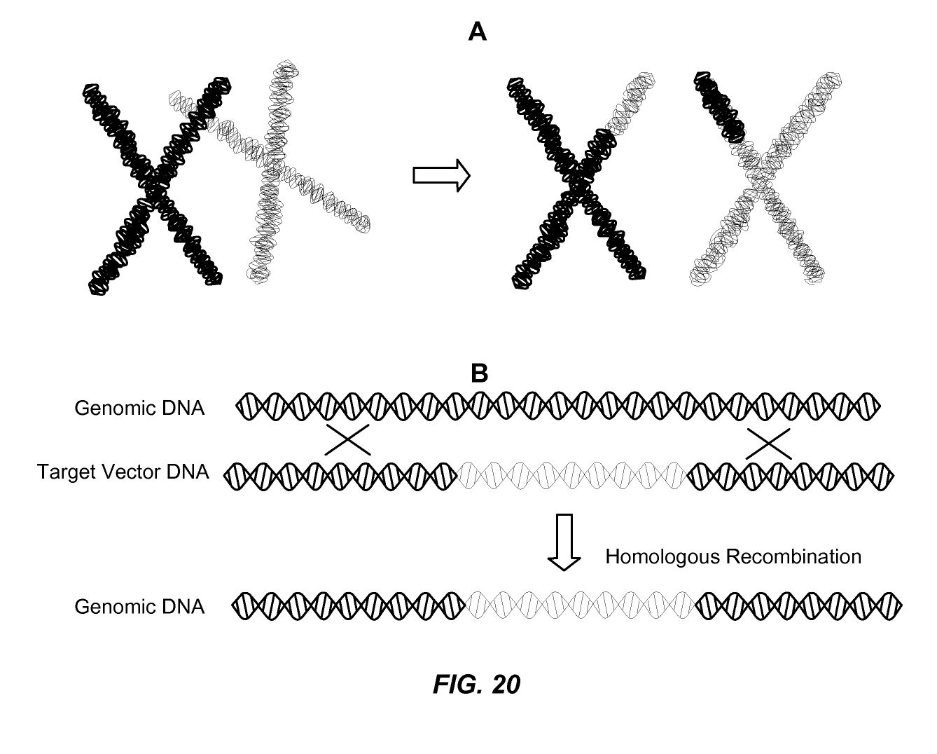

Homologous recombination itself occurs commonly during the process of meiosis in eukaryotic systems. The process involves the alignment of highly similar DNA sequences in chromosomes, and the exchange of DNA sequences between the DNA in each of the sister chromosomes. The complex series of molecular interactions is simply defined as “cross-over” (FIG. 20A). When these sequences are aligned, breaks in the double strand of DNA facilitate the swapping of genetic material. Two homologous sequences flanking a non-homologous sequence can be used to introduce a foreign DNA fragment to the genomic DNA (FIG. 20B). This strategy has been used extensively for gene knock-in or knock-out in mice. While there are several potential advantages of this system, the main one is the elimination of the foreign or heterologous DNA randomly inserting into the DNA. Similarly, because homologous recombination requires highly similar stretches of DNA sequence, one can be relatively certain of the location of the integrated insert and its copy number (one or two as compared with 3-6 for retroviral delivery).

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Materials and Methods

### iPS Generation and Culture

Normal fibroblasts from Green Fluorescence Protein (GFP) transgenic mice (C57BL/6-Tg(UBC-GFP)30Scha/J Stock #004353) (Jackson Laboratory, Bar Harbor, Me.) were transduced with 4 retroviral transcription factors as described by Yamanaka et al. (12, 13). The newly generated undifferentiated mouse iPS cells were maintained on Mitomycin-C treated mouse embryo fibroblast feeder layers in ES medium containing Knockout DMEM (KDMEM, Invitrogen, Carlsbad, Calif.), 15% ES qualified fetal bovine serum (FBS) (PAA, Ontario, Canada), 2 mM L-Glutamine (Invitrogen), 1×10⁻⁴ M nonessential amino acids (Invitrogen), 1×10⁻⁴ M 2-mercaptoethanol (Sigma, St. Louis, Mo.), 1× pen/strep (Invitrogen) and 12.5 ng/ml LIF (Chemicon, Billerica, Mass.). Cultures were passaged using 0.25% trypsin (Invitrogen) at a 1:3-1:6 split ratio every 3-4 days. Mouse embryo fibroblast feeder cells (Millipore, Billerica, Mass.) were maintained in DMEM High Glucose supplemented with 10% certified FBS (Invitrogen) and 1× pen step.

### Induction of iPS Cell-Derived Pancreatic Beta-Cells

The 3-stage differentiation protocol of iPS cells into β-cells was adapted from the differentiation of embryonic stem (ES) cells into insulin-producing cells by Wobus et a with modification (17). At stage 1, embryoid bodies (EB) were generated from iPS cells. iPS cells attached to feeder layers in 10 cm tissue culture dishes were washed one time with 1× PBS (Invitrogen) to remove residual serum. The cells were treated with 0.25% trypsin for 30 seconds, trypsin was removed and cells were disrupted by manually scraping the culture dish with a sterile cell scraper (BD Biosciences). EB medium (ES medium without LIF) was added to the cells and the cells were further disrupted into single cells and small pieces by pipetting up and down. The cell suspension was centrifuged at 300 g for 5 min. The supernatant was removed and cell pellet was resuspended into 10 ml of EB medium. To deplete iPS cells of unwanted feeder cells, cells were plated onto a T75 tissue culture flask and the feeder cells were allowed to attach for 30-45 minutes at 37° C. in a humidified CO₂ incubator. The feeder-depleted cells were then collected and centrifuged at 300 g for 5 min. The cell pellet was resuspended in EB media at 6000 cells/mL and plated as drops on 15 cm Petri dishes at 18-22 rows of drops per plate. Plates were gently flipped to invert drops and incubated at 37° C. incubator for 2 days. The EBs were collected and pooled by gently swirling plates with 1× PBS and transferred to a 50 mL conical tube. Pooled EBs were allowed to settle by gravity (about 10 min.), resuspended in 10 mL EB medium, transferred to 10 cm non-adherent petri plates and incubated for 3 more days.

At stage 2, EBs were collected and transferred to adherent culture at 10-15 EBs/10 cm tissue culture dish to induce multi-lineage progenitors. For RT-PCR, immunofluorescence staining and ELISA assay, 1-3 EBs were transferred into each well of 12-well plates. The EBs cultured in EB medium were differentiated for 9 days, with media changes every 3-4 days.

Finally, at stage 3, EBs were induced to further differentiate into β-like cells for 7-20 days. EB medium was replaced with β-cell selective differentiation medium consisting of DMEM/F12 supplemented with 10% Knockout replacement serum, 20 nM progesterone (Sigma, St. Louis, Mo.), 100 μM putrescine (Sigma), 1 μg ml-1 laminin (Sigma), 10 mM nicotinamide (Sigma), 1× ITS premix containing insulin, transferrin and selenic acid (BD Biosciences), 1327 media supplement (Invitrogen), and 1× pen/strep. After day 6 in selective medium, cells were trypsinized and replated into new culture dishes. Again, media was changed every 3-4 days throughout stage 3.

### In Vitro Insulin Induction and ELISA Assay

Stage 3, day 7, 13, and 20 iPS cell derived β-like cells grown to ˜80-90% confluence in 12-well multiwell plates were washed three times with 1× PBS. The wash was removed, 500 μl of KRBH solution (KRBH: 129 mM NaCl, 4.8 mM KCl, 2.5 mM CaCl₂, 1.2 mM KH₂PO₄, 1.2 mM MgSO₄, 5 mM NaHCO₃, 10 mM HEPES, 0.1% (wt/vol) BSA) with 2.5 mM glucose was added and the cells were incubated at 37° C. In a humidified CO₂ incubator for 90 min. The cells were then washed 3× with PBS to remove residual insulin produced during the initial induction. KRBH solution containing 5 mM, 10 mM, 20 mM, 30 mM or 40 mM glucose was added and the cells were returned to the incubator for 90 min. Supernatants were collected and saved, the cells were again washed 3× with 1× PBS, and 500 μl of KRBH solution (no glucose) was then added to the cells to test whether the β-like cells continue to produce insulin in the absence of glucose. The levels of insulin produced by in vitro derived cells were measured using an ELISA based insulin assay (Mercodia, Upsala Sweden) performed according to manufacturer's instructions.

### Fluorescence Activated Cell Sorting, FACS

Prior to cellular transplantation, stage 3, day 7 iPS cell derived β-like cells from two 10 cm culture dishes were dissociated into single cell suspensions by trypsinization. The cells were washed once with 1× PBS and stained with SSEA-1 antibody (1:200 antibody dilution in 5 ml 1× PBS, see Table 2) for 30 min. at RT. SSEA-1 is a surface antigen found on stem cells and is used extensively for excluding cells bearing stem cell markers to reduce the chance of teratoma formation after transplantation. The cells were pelleted by centrifugation at 300 g for 5 min. The cells were stained with secondary antibody (1:1000 antibody dilution in 5 ml 1× PBS, Table 2) and incubated for 15 min, at RT. The cells were washed three times with 1× PBS and resuspended in β cell differentiation medium at a concentration of 2.5-5×10⁶ cells/ml medium. GFP^{positive}/SSEA1^{negative} cells were sorted using an iCyt Reflection cell sorter. The sorted cells were washed once with 1× PBS and resuspended into basal DMEM/F12 at a concentration of about 600 cells/μl prior to transplantation.

### Diabetes Mellitus Mouse Models

All animal procedures were approved by Applicants' institutional animal committee. The Type 2 diabetes mellitus mouse model, Lepr^{db}, D57BLKS; Dock7^{m}, DBA/J was purchased from Jackson Laboratories (Stock #000642). We used genotyped mice homozygous for the spontaneous diabetes mutation (Lepr db), which causes these mice to become obese at age 3-4 weeks. The mice also become hyperglycemic at 3-4 weeks of age due to unregulated glucose metabolism and severe destruction of insulin-producing pancreatic β cells. As the mice continue to age, they become resistant to exogenous insulin therapy (21, 22).

For the Type 1 diabetes mellitus mouse model, C57BL6 mice were given a single dose of 180 mg/kg of streptozotocin, STZ, (Sigma) via intraperitoneal injection. STZ is a chemical toxic to pancreatic β cells. The diabetic phenotype was confirmed by hyperglycemic readings. This diabetic condition was allowed to stabilize for 10 days prior to cellular transplantation. For these studies, STZ-treated mice with fasting glucose concentrations of >400 mg/dl were used.

Insulin Tolerance Test

Insulin tolerance test was performed as previously described (23). Untransplanted Type 2 female diabetic mice (fasted for 6 hours) at 4, 8 and 16 weeks of age (n=3 per age group) were given 0.75 U/kg of human insulin (Novo Nordisk, Clayton, N.C.) via intraperitoneal injection. Blood glucose readings were obtained at 30, 60, and 90 minutes post injection via the tail vein using a hand-held LifeScan OneTouch glucometer (Johnson &. Johnson, New Brunswick, N.J.).

Cellular Therapy of Diabetes Mellitus Mouse Models

Cellular transplantation was done by portal vein injection using previously established protocols (24). Under anesthesia, 200,000 FACS-sorted GFP^{positive}/SSEA1^{negative} stage 3 iPS cell derived β-like cells in 300 μl basal DMEM/F12 medium were injected into the hepatic portal vein using a 32 gauge Hamilton syringe. Intraportal vein injection of in vitro derived β-like cells is an efficient way to directly engraft the cells into the murine hepatic sinusoids. The engrafted cells stably expressed green fluorescence protein enabling us to recognize and distinguish the transplanted cells from other hepatic cell types. Glucose readings were obtained every 2-3 days post transplantation from fasted mice via the tail vein using a hand held glucometer. 5 μl of blood were collected from three transplanted mice with normal glucose readings 4 weeks post transplantation (n=3) and the samples were sent to an independent company (Diabetes Technologies, Inc.) to obtain the hemoglobin Alc readings.

### Statistics

Statistical differences between groups (P<0.05) were determined by Excel's one-tail student t-test. P value less than 0.05 was considered statistically significant.

### RT-PCR

Total RNA was isolated from undifferentiated iPS cells and differentiated β-like cells using Trizol reagent (Invitrogen) according to manufacturer's instruction. 2 μg of total RNA was used for reverse transcription with cDNA Archive kit (Applied Biosystems, Foster City, Calif.) according to manufacturer's instruction. PCR reactions were performed using Platinum PCR Supermix High Fidelity (Invitrogen) according to manufacturer's instructions. PCR primer sequences for endogenous/total retroviral transcripts (8) and specific pancreatic markers (25) are provided Table 1.

### Teratoma Formation in Nude Mice

Evaluation of proliferation and differentiation of iPS cells was tested via teratoma formation in nude mice. iPS cells were dissociated with 0.25% trypsin for 2 minutes and then manually scraped using a cell scraper, collected and transferred into 15 ml conical tube, centrifuged at 1500 RPM for 5 minutes, and the cell pellet was resuspended in EB differentiation medium. 2×10⁶ dissociated iPS cells in 100 μl DMEM High Glucose were mixed with an equal volume of Geltrex (Invitrogen) and injected subcutaneously into flanks of 2 nude mice (Taconic, Hudson, N.Y.), Thirty days post injection, teratomas were removed, dissected and fixed with formalin (Fisher Scientific, Pittsburgh, Pa.). Paraffin-embedded tissues were sectioned and stained with hematoxylin and eosin.

### Immunofluorescent and Tissue Staining

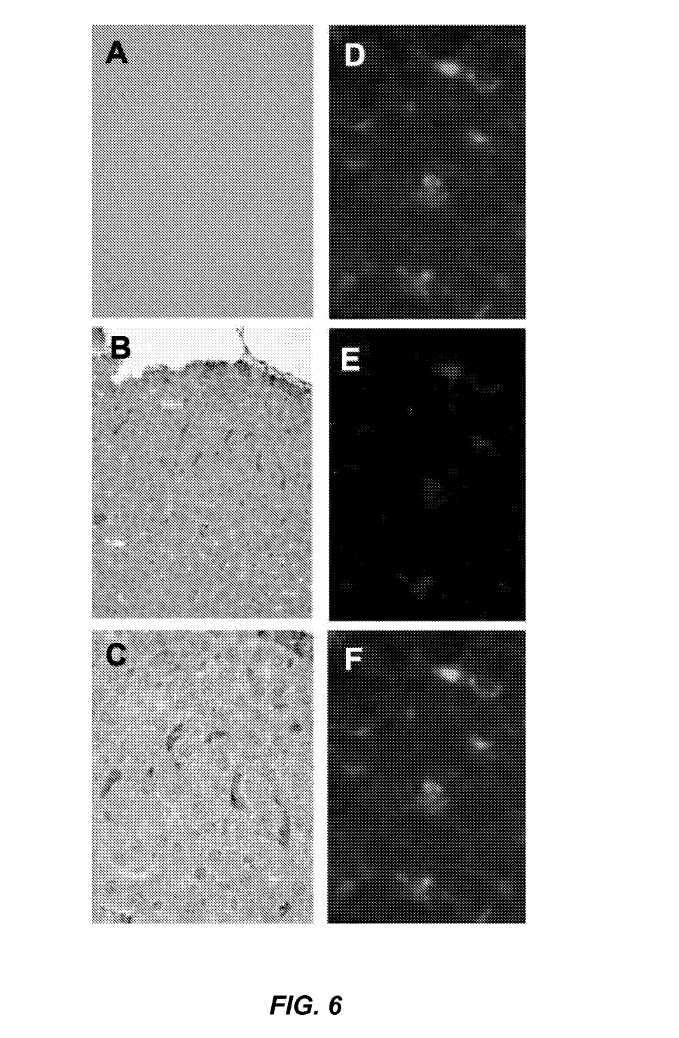

Cell cultures were washed three times with 1× PBS and fixed for 15-30 minutes at room temperature (RT) in 4% paraformaldehyde in PBS (EMD Chemicals, Gibbstown, N.J.). They were then washed 3× in PBS, and permeabilized with 0.2% Triton X (Fisher Scientific, Pittsburgh, PA) in PBS for 15-30 minutes at RT. For surface markers such as SSEA-1, the permeabilization step was omitted. The cells were again washed 3× with PBS and blocked for 15-30 min. in 5% bovine serum albumin (Fisher Scientific) in PBS at RT. Primary: and secondary antibodies are listed in Table 2. Primary antibodies in 1% BSA were added to the cells and incubated for 2 hrs. at 37° C. The cells were washed again 3× with PBS, and secondary antibodies diluted in PBS were added and incubated for 1 hour at 37° C. The cells were washed again and the nuclei were counterstained with 1 mg/nil 4′,6-diamidino-2-phenylindole DAPI (Sigma, St. Louis, Mo.) for 10 minutes followed by a final 3 washes in PBS. Liver tissues were prepared by either of two methods, paraffin embedded tissues were used for the immunohistochemistry shown in FIG. 6, panels A, B, and C. Frozen sections were prepared for the fluorescent images in in FIG. 6, panels D, E and F.

Livers were isolated and fixed in 10% neutral-buffered formalin and embedded in paraffin. 5 micron sections were cut, placed on pre-cleaned slides, deparaffinized using xylene, and then hydrated using an alcohol gradient. IHC staining using rabbit anti-mouse GFP primary antibody was performed using RMR622G Rabbit HRP Polymer kit (Biocare, Concord, Calif.). Deparaffinized slides were placed in 1× Rodent Decloaker solution and heated to 125° C. for 30 min. using Biocare's Decloaking Chamber. The slides were removed and washed 3× with deionized water. Rodent Block M was applied for 30 min. to reduce both non-specific background staining and endogenous mouse IgG. The slides were washed 2× with PBS, and GFP primary antibody in 1% antibody dilution buffer (1:100) was applied and incubated overnight at 4° C. in a humidified chamber. The slides were washed with PBS and Rabbit HRP Polymer solution was applied for 20 nun. The slides were again washed in PBS and Betazoid DAB Chromogen Reagents (1 drop of Betazoid DAB Chromogen added to 1 ml of Betazoid DAB Substrate Buffer) was applied for 5 min. The slides were then rinsed with deionized water and treated with hematoxylin for 30 seconds. The slides were washed with warm alkaline tap water to blue the nuclei. The slides were then dehydrated, cleared and coverslipped. Cytoplasm appears brown and nuclei appear blue.

5 μm frozen liver sections were placed on positively charged slides and allowed to air dry at RT for 5 min. before fixing with 3% formaldehyde/methanol for 15 min. at RT, followed by 5 min. incubation in methanol at −20° C. The sections were washed 2× with PBS for 5 min. and incubated for 10 min. at RT in 3% hydrogen peroxide diluted in methanol. The sections were washed 2× with PBS for 5 min and then incubated with 5% BSA blocking buffer for 1 hour at RT. The blocking buffer was removed and 400 μl of insulin monoclonal primary antibody (1:100, see Table 2) in 1% BSA as added and incubated overnight at 4° C. The antibody solution was removed and the sections were washed 3× with PBS for 5 min. each. Secondary antibody diluted 1:1000 with PBS was then added and incubated for 30 min. at RT. The secondary antibody solution was removed and the sections were washed 3× in PBS for 5 min. each. Nuclei were stained with 1 mg/ml DAPI for 10 min. and sections were washed 3× with PBS for 5 min. each. Images were immediately captured on a fluorescent microscope.

### Example 2

Reprogramming of Normal Mouse Skin Fibroblasts into Induced Pluripotent Stem Cells

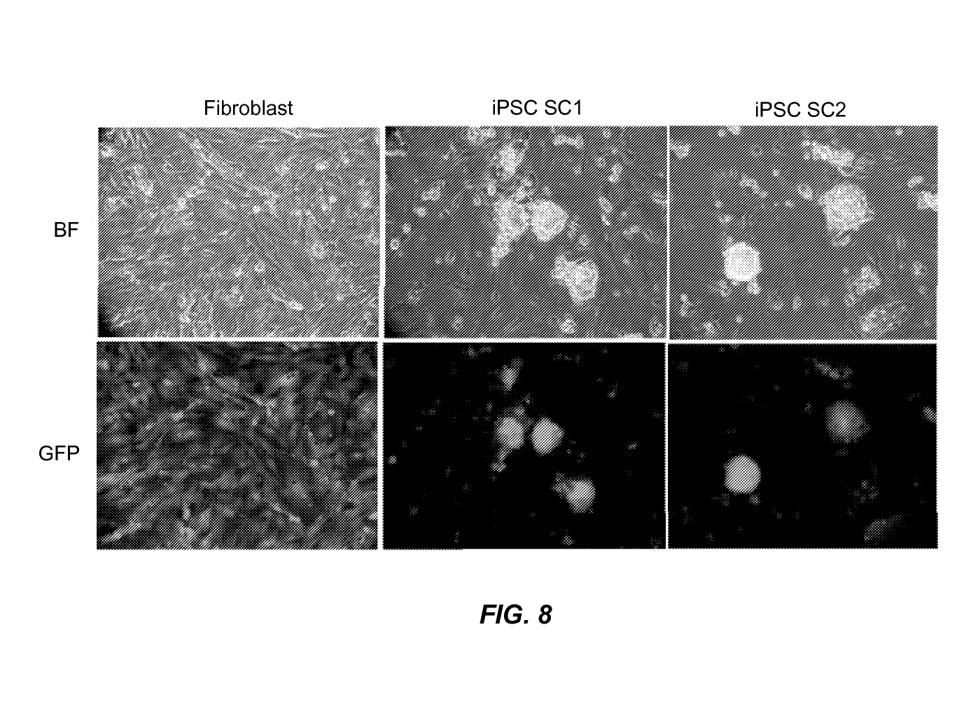

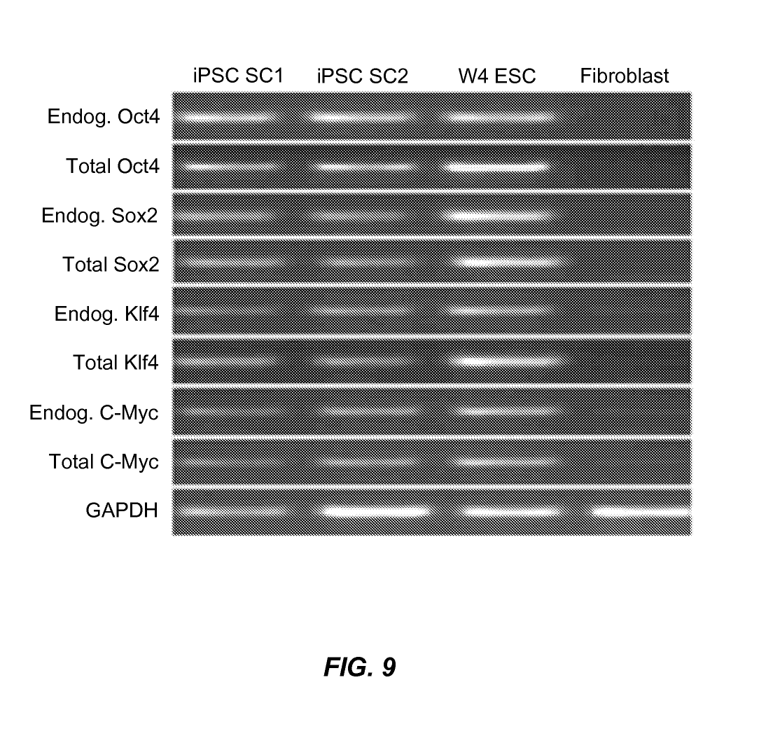

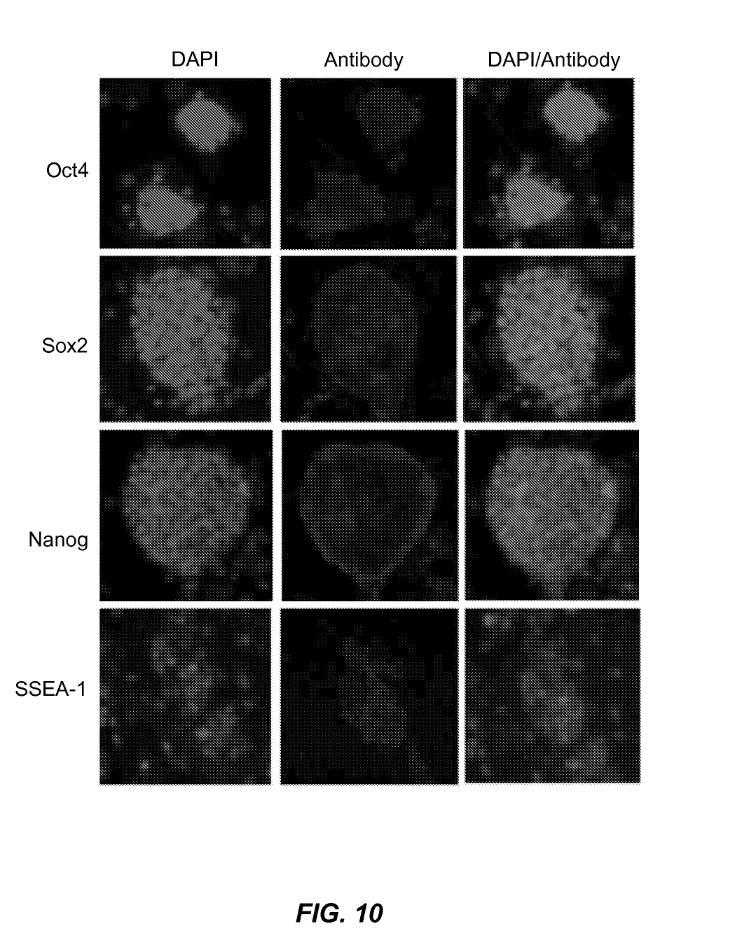

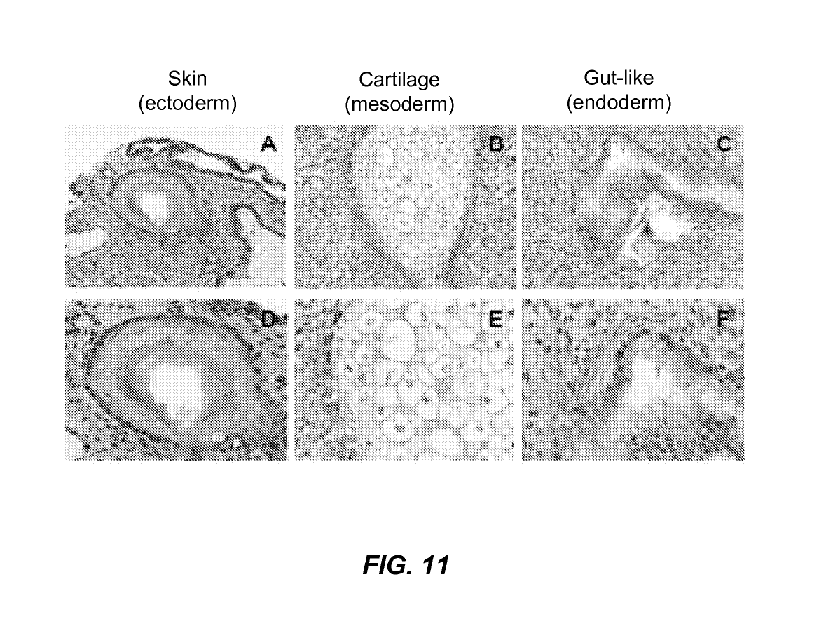

Normal fibroblasts from Green Fluorescence Protein ((T) transgenic mice were transduced with 4 retroviral transcription factors (10-12), using methods that we have previously described (26). In this current study, two iPS subclone colonies were picked and expanded at 20 days post transduction (FIG. 8). mRNA expression profiles of these two iPS subclones show upregulation of the endogenous stem cell markers Oct4, Sox2, Klf4, and c-Myc similar to those of embryonic stein (ES) cells (FIG. 9). There was minimal expression of exogenous viral transgenes exhibited. Immunofluorescence staining, used to access surface and intracellular antigens present in these iPS cells, was positive for the well established pluripotent markers, Oct4, Sox2, Nanog and SSEA-1 (FIG. 10). To determine their capacity for proliferation and differentiation, roughly 2×10⁶ dissociated iPS cells were injected subcutaneously into the flanks of three nude mice. Thirty days post injection, a teratoma had formed in all injected mice, suggesting full proliferative capacity of the injected cells. Differentiation was established via hematoxylin and eosin staining of tissue from the teratomas, showing the presence of cell types belonging to the three germ layers: ectoderm (skin), endoderm (gut-like) and mesoderm (cartilage) (FIG. 11). The morphology, mRNA expression profile, immunofluorescence staining and teratoma formation confirm the in vitro generation of ES-like iPS cells.

### Example 3

Selective Differentiation of iPS Cells into Insulin-Producing Pancreatic β-Like Cells

iPS cells were driven to undergo a 3 stage differentiation into β-like cells which can produce insulin using modification of a previously published protocol for ES cells (17). The time line of this differentiation is shown in FIG. 1. At stage 1, the ES-like iPS cell cultures were dissociated into single cells and placed in suspension culture where they form embryoid bodies (FIG. 1, top panel). After 5 days in suspension culture, the embryoid bodies were returned to adherent culture to undergo further differentiation for 9 days. During this time, cells migrated from the attached spheroids (FIG. 1, center panel). After 9 days, the media was replaced with selective media containing laminin, insulin, nicotinamide, selenic acid, transferrin, progesterone and Knock-Out replacement serum, marking the beginning of stage 3. During the ensuing 20 days the cells continued to differentiate (FIG. 1, bottom panel). The prior protocol (17) had included enzymatic detachment of the differentiated cells at the end of stage 2. This procedure was detrimental to the differentiating pancreatic cells, with only 10% of the cells surviving and reattaching after trypsinization. Therefore, after 9 days, replaced the stage 2 medium with stage 3 selective medium without trypsinizing, and cell survival increased by 80%.

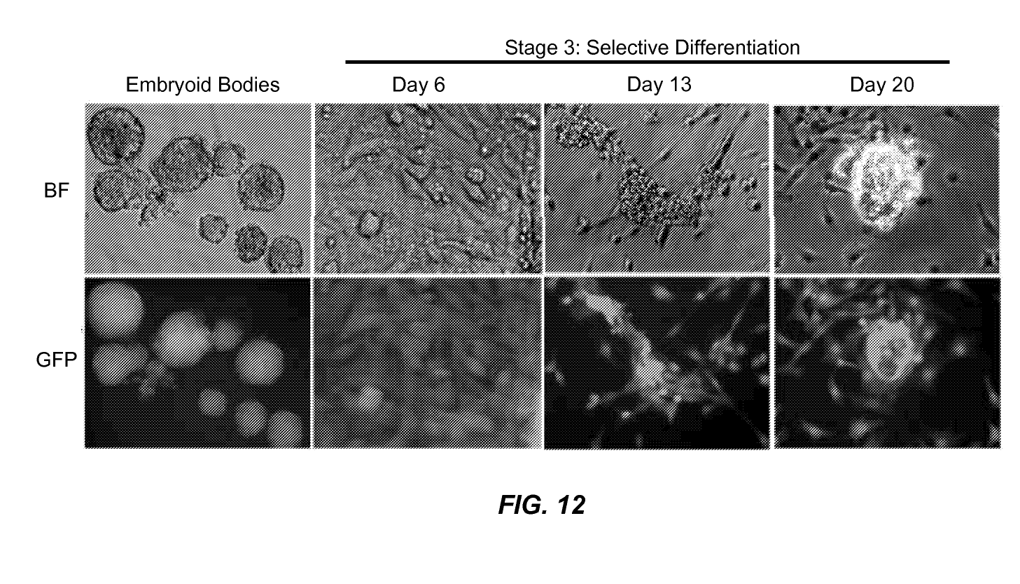

During stage 3 differentiation, multi-lineage cells were seen to he present at day 6 of selective media treatment (FIG. 12, column 2). At this time, the cultures were trypsinized and replated onto new dishes in fresh selective medium. On Day 13, numerous clusters began to form (FIG. 12, column 3). By day 20, large cell clusters were evident (FIG. 12, colummn 4). GFP was highly expressed throughout the differentiation process.

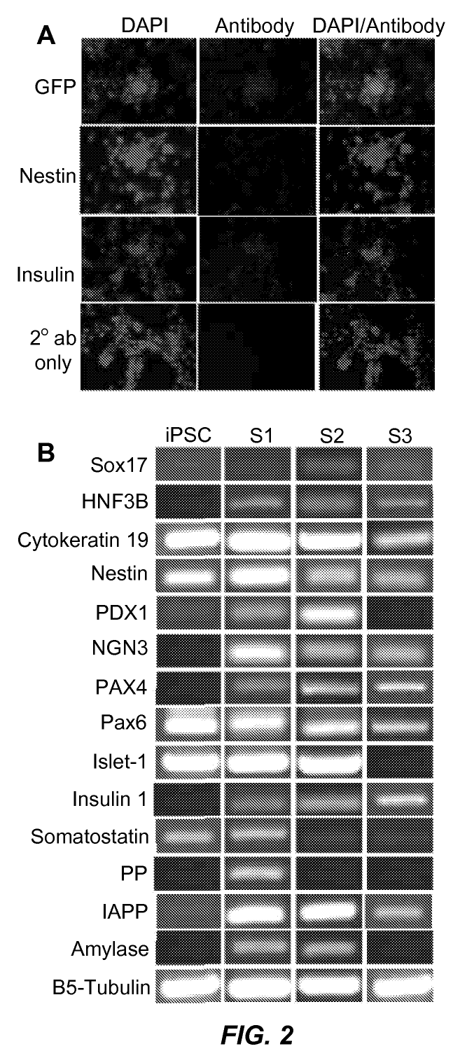

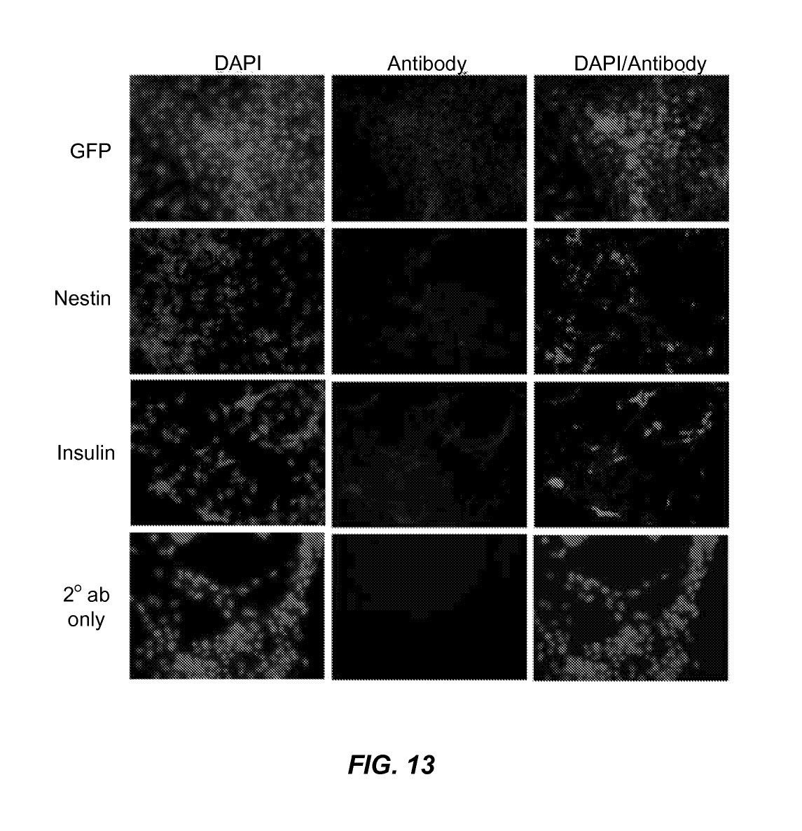

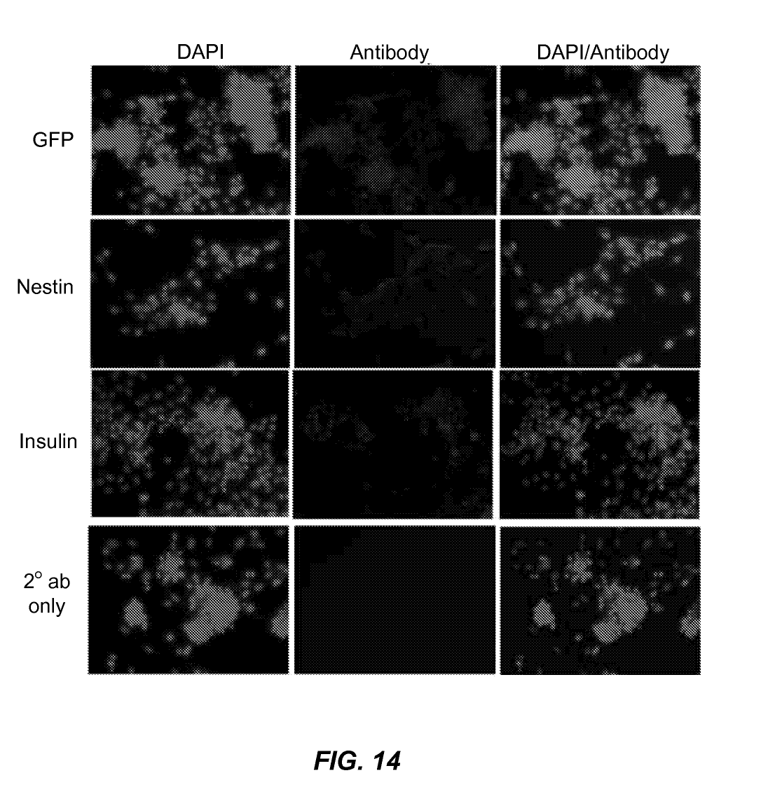

Cellular levels of insulin and nestin were analyzed at stage 3 by immunofluorescence staining. At day 7, both insulin and nestin are expressed (FIG. 13). Expression of nestin peaked at day 13 (FIG. 14) and decreased by day 20 (FIG. 2A). Insulin was expressed at low levels at days 13 and 20 consistent with previous reports (7).

mRNA analysis revealed expression of several specific markers of in vitro pancreatic β cell differentiation as shown in FIG. 2B. The definitive endoderm markers, Sox17 and HNF3B (FoxA2) were co-expressed in stage 2 and were detectable up to stage 3. Co-expression of cytokeratin 19 and nestin, suggesting the presence of multi-lineage progenitors, is seen during stages 1 through 3. Co-expression of PDX1 and HNF3B, indicative of pancreatic endoderm or epithelium generation, is seen in stages 1 and 2. NGN3, highly expressed in all endocrine progenitors (18), is co-expressed with PDX1 in stages 1 and 2, indicating further commitment of the differentiated cells to the endocrine lineage. Pax4, Pax6 and Islet-1 are important transcription factors controlling endocrine cell differentiation and were detected in all 3 stages. Islet-1 is, however, downregulated early in stage 3 as the cells progressed into a mature, differentiated phenotype.

There are 5 endocrine cell types, α, β, δ, PP and ε cells, which produce the hormones glucagon, insulin, somatostatin, pancreatic polypeptide (PP) and ghrelin, respectively. Insulin expression was detected as early as stage 1 and remained upregulated until stage 3. Furthermore, islet amyloid polypeptide (IAPP), secreted by pancreatic β-cells at the same time as insulin (19, 20), was also highly expressed in stages 1 to 3. Expression of somatostatin and PP was only evident in stage 1. Glucagon was either undetectable or had low expression in all of the differentiation stages (data not shown). Ghrelin was not tested. Amylase positive-acinar cells were detected in stages 1 and 2 but were undetectable at the selective differentiation stage 3.

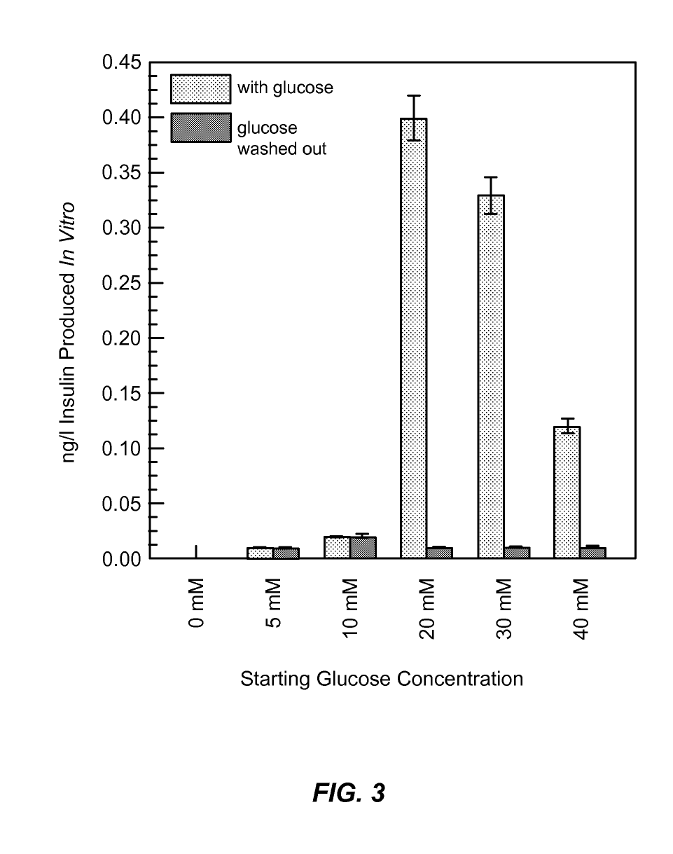

Glucose responsiveness of iPS derived β-like cells was tested in vitro by exposure of glucose starved stage 3 cells to 5 glucose concentrations for 90 minutes. As shown in FIG. 3, day 20 differentiated cells responded to glucose in a dose-dependent manner. At the lower glucose concentrations (5 mM and 10 mM), the differentiated cells were marginally responsive. At 20 mM glucose, insulin production peaked, at ˜8 fold higher than readings from 5 mM and 10 mM glucose-treated cells. At 30 mM and 40 mM, the insulin readings begin to decrease again. Following glucose induction, the cells were washed thoroughly to remove residual insulin, and then re-incubated with basal KRBH solution for 90 minutes. As shown in the same graph, no insulin was produced when glucose is removed, suggesting that the in vitro derived β-like cells will only produce insulin in response to glucose treatment. Insulin was also produced by day 7 and 13 differentiated cells when exposed to glucose treatment (data not shown).

### Example 4

### Type 2 Diabetic Mouse Model Phenotypic Tests Reveal Hyperglycemia, Abnormal Insulin Levels, and Resistance to Exogenous Insulin Therapy

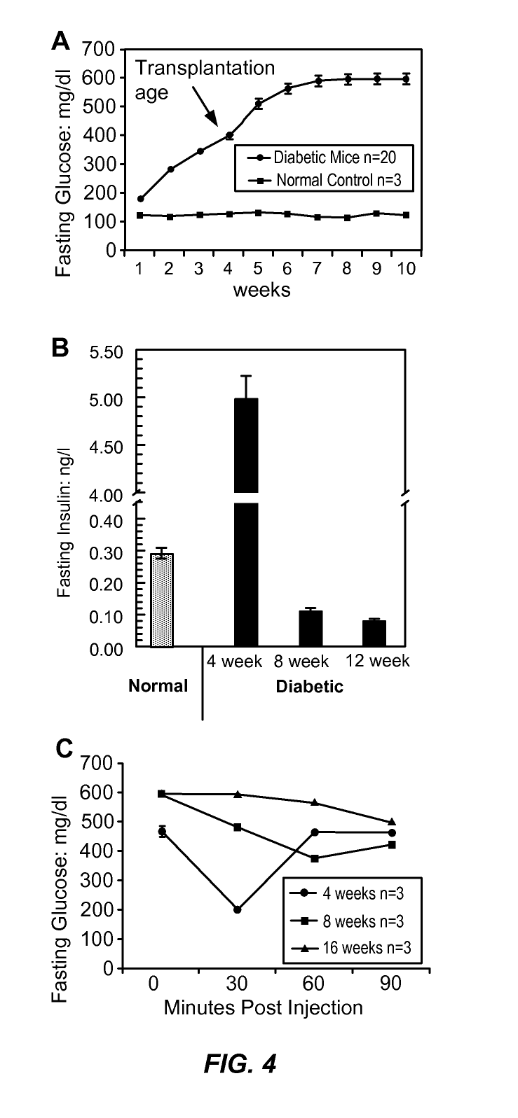

The Type 2 diabetes mouse model (Lepr^{db}, C57BLKS; Dock7^{m}, DBA/J) used herein shows depleted insulin production as the mice age, while maintaining a hyperglycemic phenotype. By the age of 3-4 weeks diabetic mice demonstrated hyperglycemia after 6-8 hours of fasting, with glucose concentrations greater than 300 mg/dl as compared to the normal C57BL6 mouse strain used as a control (FIG. 4A). At 3 weeks of age, diabetic mouse blood glucose concentrations (n=20) increased ˜2.6 fold as compared to normal controls. At 4-5 weeks of age, the glucose concentration of the diabetic mice increased ˜3.8 fold. After 6 weeks of age, the hyperglycemia is very severe and glucose levels are dangerously high, at >600 mg/dl.

Insulin levels in serum samples collected from 4 week old diabetic mice (n=20) (FIG. 4B), were highly variable, ranging from 0.64 to 15.32 The mean insulin level was ˜17 fold higher than that of normal control mice n=3). As the diabetic mice continued to age, insulin levels decreased. Diabetic mice at 8 weeks (n=3) had ˜2.6 fold lower insulin levels and at 13 weeks (n=3) had ˜3.6 fold lower insulin levels than the normal controls.

Insulin resistance also developed as the mice aged (FIG. 4C). Exogenous insulin, injected into 4 week old (n=3) diabetic mice led to a drop in fasting glucose, occurring between 0 to 30 minutes post injection. With increased age the mice, at 8 weeks (n=3) and 16 weeks (n=3), showed minimal response to the injection, indicating resistance to insulin.

### Example 5

iPS Derived β-Like Cells were Able to Engraft in Liver Parenchyma and Ameliorate Hyperglycemia in the Type 2 Diabetes Mouse Model

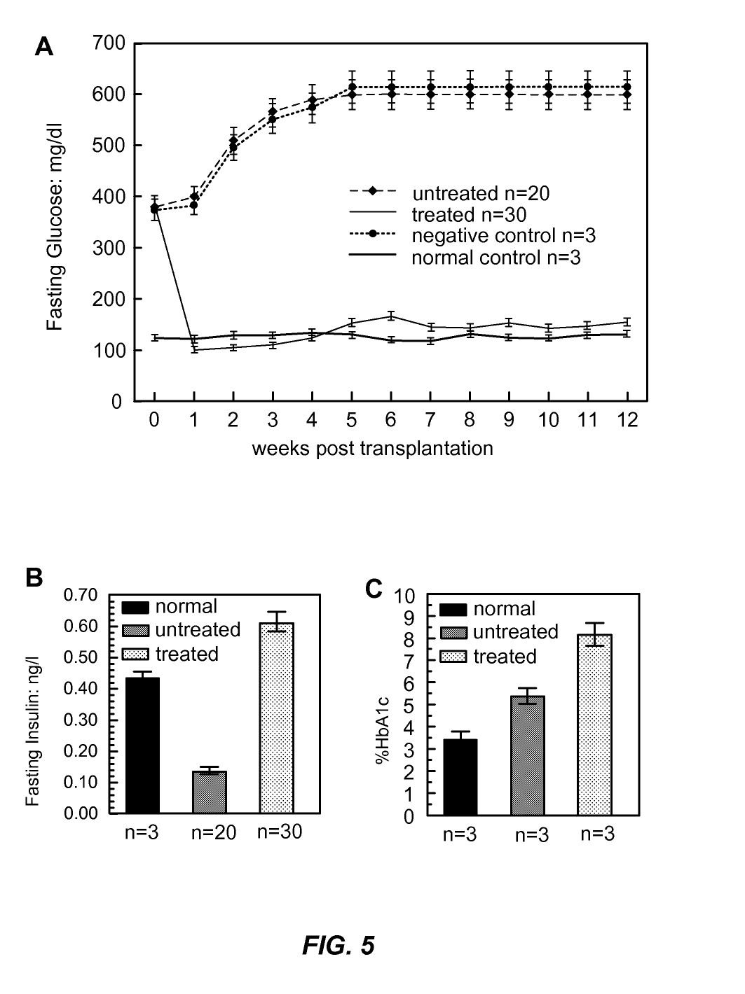

iPS derived insulin-secreting β-like cells were isolated by FAGS sorting to yield GFP^{positive}/SSEA1^{negative} cells which were transplanted into diabetic mice (n=30) by intraportal vein injection. Fasting glucose measurements, begun 2 days post-transplantation, are shown in FIG. 5A. All 30 transplanted mice (exhibiting 100% engraftment efficiency) exhibited normal glucose concentrations with readings comparable to C57BL6 normal controls (n=3). Three diabetic mice transplanted with GFP^{negative}/SSEA1^{negative} negative control cells, (feeder cells), remained hyperglycemic. The unengrafted diabetic counterparts (n=20) remained hyperglycemic as well.

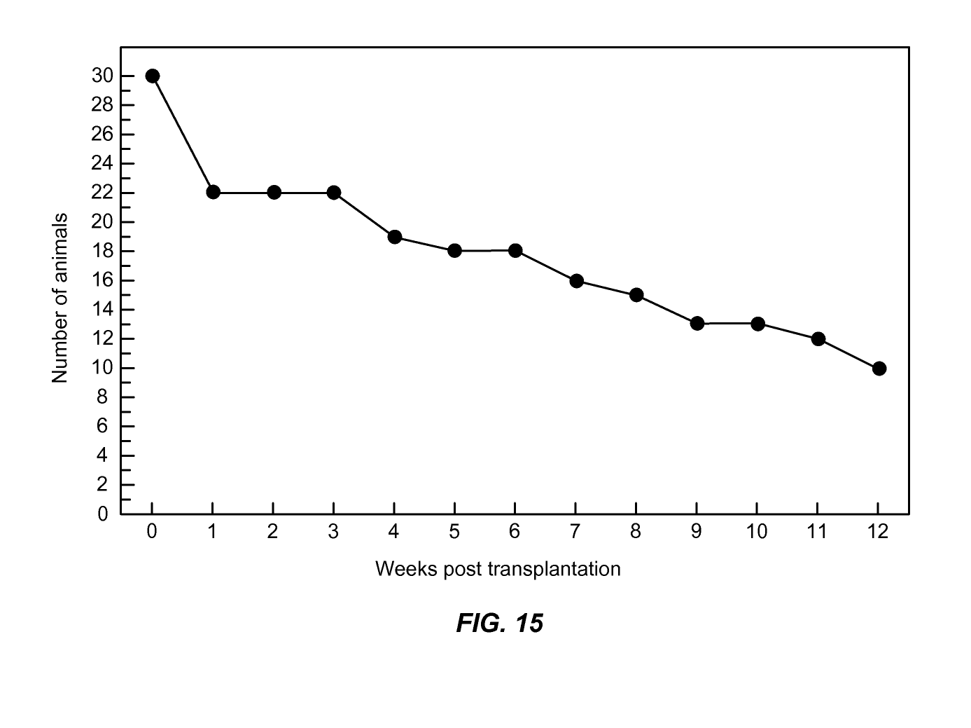

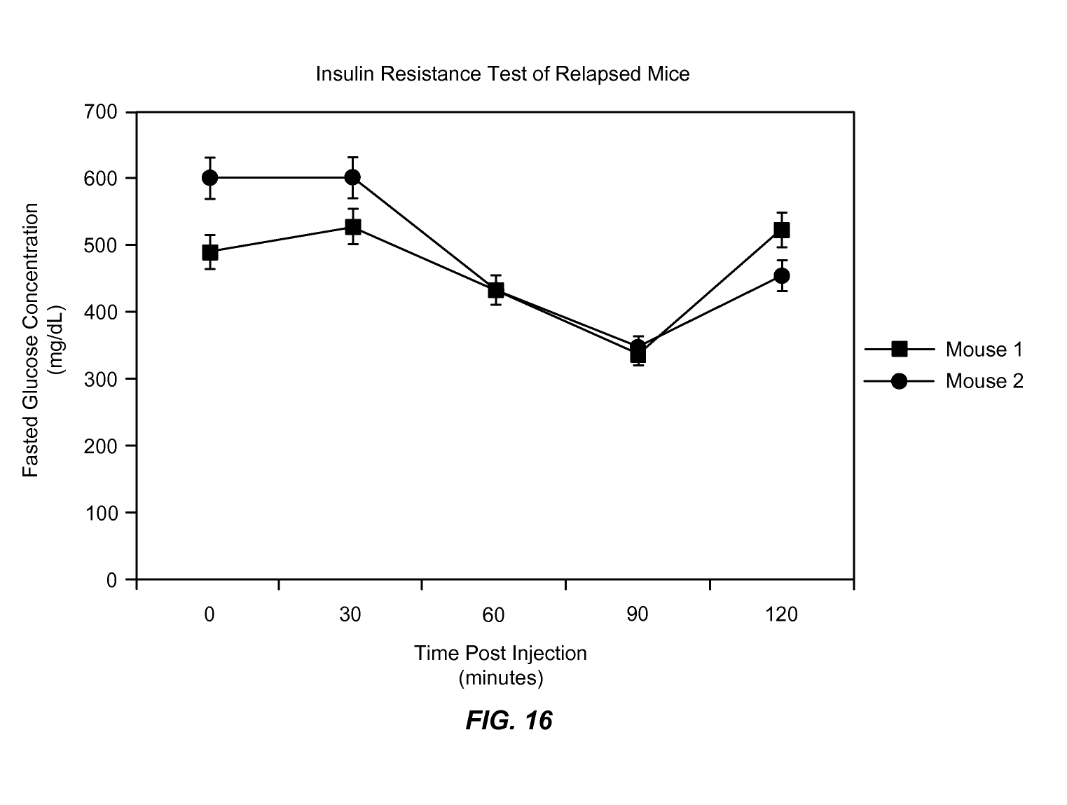

At 4 weeks post transplantation, 20 of the original 30 β□-like cell transplanted mice survived and maintained normal glycemic control. At 8 weeks post transplantation, 15 of these mice were still alive and normoglycemic. Data to week 12 showed continued maintenance of normal glucose levels in the treated mice. The 50% mortality rate in the transplanted mice (FIG. 15) appeared to be the result of complications (i.e., thrombosis) resulting from the surgical access of the portal vein. However, all of the mice survived for at least 1 week post-transplantation and exhibited normal glucose levels during that time. Two mice had relapsed into hyperglycemia after maintaining regulated glucose level for 8 weeks. These 2 mice appeared to have re-developed insulin resistance (FIG. 16).

### Example 6

Engrafted iPS Derived β-Like Cells were Able to Produce Insulin In Vivo, and Normalized Hemoglobin Alc Levels

The amelioration of hyperglycemia in the Type 2 diabetic mouse model transplanted with iPS cell derived β-like cells occurred concomitantly with an increase in in-vivo insulin concentration measured by mouse insulin ELISA assays. By 21-56 days post transplantation β-like cell transplanted mice had ˜4.35 fold increase in insulin levels as compared to untreated mice (P<0.05) (FIG. 5B). Insulin levels in the treated mice were somewhat higher than those of the untreated controls.

Hemoglobin Alc tests to measure the amount of glucose attached to the hemoglobin of red blood cells were done 4 weeks post transplantation (FIG. 5C)

The hemoglobin Alc levels of untreated diabetic mice (n=3) were ˜2.4 fold higher than normal C57BL6 controls (n=3). Transplanted mice with normal glucose readings 4 weeks post transplantation (n=3) had significantly improved the hemoglobin Alc readings (a better than 70% improvement over the untreated mice).

### Example 7

### Cell Distribution of Engrafted Cells in the Liver Parenchyma

Intraportal vein injection of in vitro derived β-like cells is an efficient way to directly engraft the cells into the murine hepatic sinusoids. At 7 days and 4 weeks post transplantation, 3 mice were sacrificed to obtain tissues for immunohistochemical and immunofluorescence analysis to assess the distribution of engrafted cells in the liver. The engrafted cells stably expressed green fluorescence protein allowing recognition and the ability to distinguish the transplanted cells from other hepatic cell types.

As shown in FIG. 6, cells were able to engraft stably into the liver parenchyma of the diabetic mouse model. Spindle-shaped GFP positive cells detected by anti-GFP antibodies (brown) were evenly distributed throughout the microscopic liver sections of 7 day treated mice (Panels B and C). Minimal GFP positive cells were detected in lungs and spleen (data not shown). Liver sections from the C57BL6 normal mouse strain were used as negative controls (Panel A). Engrafted β-like cells co-express insulin and GFP 7 days post transplantation as detected by immunofluorescence (Panels D-F).

### Example 8

Normoglycemia in STZ Treated Mice After Transplantation with iPS Cell Derived β-Like Cells

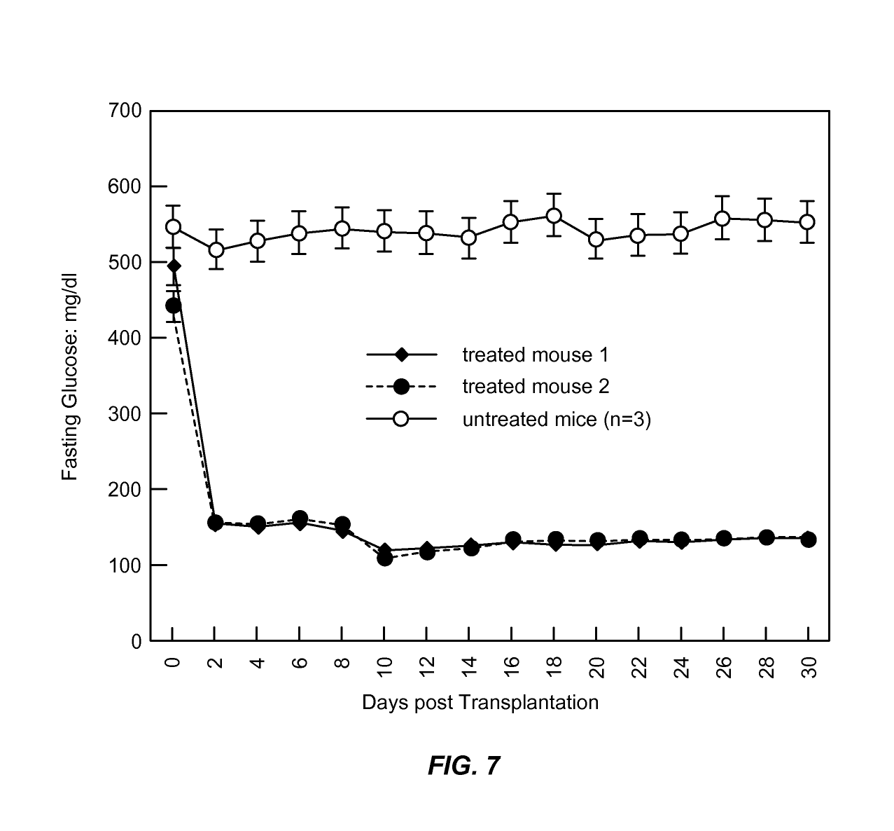

To test whether the in vitro derived β-like cells are functional in an environment where islet cells are severely depleted, a model of Type 1 diabetes, in vitro derived, insulin-secreting β-like cells were transplanted via intraportal vein injection into STZ-treated mice with glucose concentrations of >400 mg/dl (FIG. 7). At 2 days post transplantation with β-like cells, the glucose levels of the STZ-treated mice (n=2) became normal, with glucose concentrations of 160+/−5 mg/dl. Untransplanted STZ-treated mice (n=3) maintained hyperglycemia with glucose concentrations >400 mg/ell. Glucose readings of treated mice from day 2 up to day 30 post transplantation continued to be normal while the untransplanted mice remained hyperglycemic.

### Example 9

Generation of iPS Cell Without Use of c-Myc

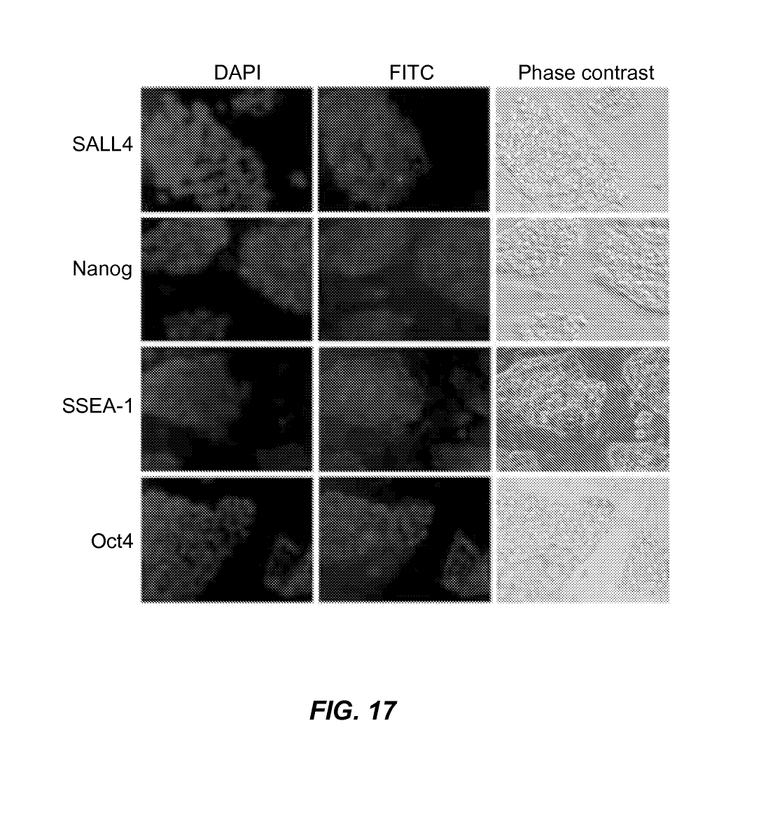

One difficulty with the clinical use of conventionally generated iPS cells is the introduction of a known oncogene, namely c-Myc, to the genome. Thus, iPS cells were generated that was devoid of this potent transcription factor. Without c-Myc, the efficiency of faithful reprogramming is observed to be much lower, but we were still able to derive iPS cells using retroviral infection using only OCT4, SOX2, and KLF4. Further characterization of these cells revealed that they express typical stem cell markers, such as SSEA-1, NANOG, and OCT4, as well as novel markers such as SALL4 (FIG. 17), suggesting that these are indeed true iPS cells. To ensure that these 3 factor iPS (3F-iPS) cells could give rise to all germ layers of the developing embryo, these cells were injected into the flanks of NOD-SCID mice. Genuine stem cells, under these conditions, will give rise to a teratoma composed of endodermal, ectodermal, and mesodermal cell lineages. Indeed, 3F-iPS cells gave rise to teratomas composed of all three germ layers within two weeks following injection. These data clearly suggest that 3F-iPS cells derived from fibroblasts have stem cell-like qualities useful for therapeutic applications.

### Example 10

### Generation of iPS Cell Using a Non-Integrating Viral Vector

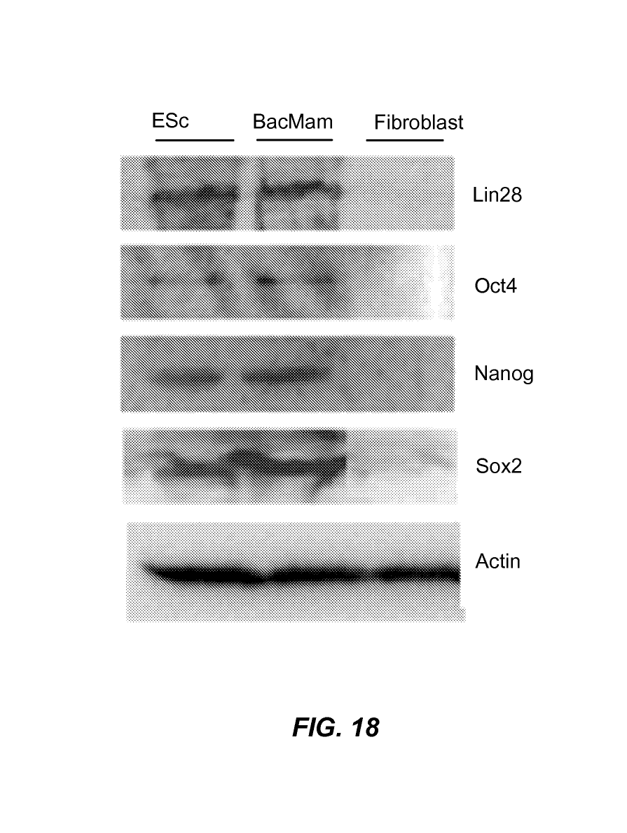

iPS cells can be produced by a new retrodifferentiation method using a baculoviral system (BacMam virus) that transiently expresses defined transcription factors in mammalian cells. The BacMan virus provides a safer approach as compared to an integrating viral vector, by eliminating accidental integration problems and offering no chance of deteriorating replication episodes since the viruses created are deleted after 2-3 cell passages. In addition, this type of virus dies away and cannot be replicated in mammalian cells, including human cells. BacMam viruses expressing Oct4, Sox2 and Nanog and Lin28 were generated. They show protein expression levels in fibroblasts that are comparable to the expression levels seen in embryonic stem cells (FIG. 18).

### Example 11

### Generation of iPS Cells Comprising Suicide Genes

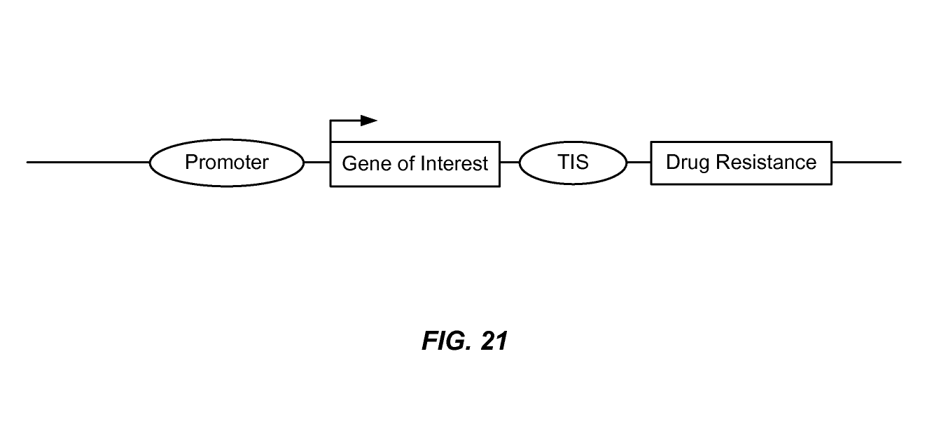

Heterologous DNA is integrated into the chromosome of the stem cell using homologous recombination to control stem cell fate after transplantation into the body. The stem cell is transformed with an expression cassette that includes an inducible promoter, the gene of interest, and a drug resistance gene. The gene of interest and the drug resistance gene should be separated by a translation initiation site (TIS; e.g., IRES) to allow for both proteins to be driven off of the same promoter (FIG. 21). By expressing the gene of interest, for example, one of the suicide genes listed above, from the cassette it would be possible to kill stem cells or reprogram the cells if these cells are harmful to the body.

The references listed below are hereby incorporated herein by reference.

### REFERENCES

1. Jones P M, Courtney M L, Burns C J, & Persaud S J (2008) Drug Discov Today 13, 888-893.
2.
3. Urban V S, Kiss J, Kovacs J, Gocza F, Vas V, Monostori E. & Uher F (2008) Stem Cells 26, 244-253.
4, Karnieli O, Izhar-Prato Y, Bulvik S, & Efrat S (2007) Stem Cells 25, 2837-2844.
5, Sun B, Roh K H, Lee S R, Lee YS, & Kang K S (2007) Biochem Biophys Res Commun 354, 919-923.
6. Lumelsky N, Blondel O, Laeng P, Velasco I, Ravin R, & McKay R (2001) Science 292, 1389-1394.
7. Docherty K, Bernardo A S, & Vallier L (2007) Semin Cell Dev Biol 18, 827-838.
8. Kroon E, Martinson L A, Kadoya K, Bang A G, Kelly O G, Eliazer S, Young H, Richardson M, Smart N G, Cunningham J, et al. (2008) Nat Biotechnol 26, 443-452.
9. Lakey J R, Mirbolooki M, & Shapiro A M (2006) Methods Mol. Biol 333, 47-104.
10. Sordi V, Melzi R, Mercalli A, Formicola R, Doglioni C, Tiboni F, Ferrari G, Nano R, Chwalek K., Lammert E, et al. Stem Cells 28, 140-151.
11. Park I H, Zhao R, West J A, Yabuuchi A, Huo H, Ince T A, Lerou P H, Lensch M W, & Daley G Q (2008) Nature 451, 141-146.
12. Lewitzky M & Yamanaka S (2007) Curr Opin Biotechnol 18, 467-473.
13. Okita K, Ichisaka T, & Yamanaka S (2007) Nature 448, 313-317.
14. Takahashi K, Tanabe K, Ohnuki M, Narita M., Ichisaka T, Tomoda K, & Yamanaka S (2007) Cell.
15. Hanna J, Wernig M, Markoulaki S, Sun C W, Meissner A, Cassady J P, Beard C, Brambrink T, Wu L C, Townes T M, et al. (2007) Science 318, 1920-1923.
16. Soldner F, Hockemeyer D, Beard C, Gao Q, Bell G W, Cook E G, Hargus G, Blak A, Cooper O, Mitalipova M, et al. (2009) Cell 136, 964-977.
17. Xu D, Alipio Z, Fink L M, Adcock D M, Yang J, Ward D C, & Ma Y (2009) Proc Natl Acad Sci USA 106, 808-813.
18. Schroeder I S, Rolletschek A, Blyszczuk P, Kania G, & Wobus A M (2006) Nat Protoc 1, 495-507.
19. Gu G, Dubauskaite J, & Melton D A (2002) Development 129, 2447-2457.
20. Hoppener J W, Oostewijk C, van Hulst K L, Verbeek J S, Capel P J, de Koning E J, Clark A, Jansz H S, & Lips C J (1994) J Cell Biochem 55 Suppl, 39-53.
21. Pittner R A, Albrandt K, Beaumont K, Gaeta L S, Koda J E, Moore C X, Rittenhouse J, & Rink Ti (1994) J Cell Biochem 55 Suppl, 19-28.
22. Brown J A, Chua S C, Jr., Liu S M, Andrews M T, & Vandenbergh J G (2000) Am J Physiol Regul Integr Comp Physiol 278, R320-330.
23. Barinaga M (1996) Science 271, 913.
24. Mathews S T, Singh G P, Ranalletta M, Cintron V J, Qiang X, Goustin A S, Jen K L, Charron M J, Jahnen-Dechent W, & Grunberger G (2002) Diabetes 51, 2450-2458.
25. Kemp C B, Knight M J, Scharp D W, Lacy P E, & Ballinger W F (1973) Nature 244, 447.
26. Takahashi K & Yamanaka S (2006) Cell 126, 663-676.
27. Xua D, Alipio A, Fink L M, Adcock D M, Yang J, Ward D C, & Ma Y (2009) PNAS 106, 808-813.

TABLE 1
Primer Sequences used for RT-PCR

| | Forward Primer | SEQ ID | Reverse Primer | SEQ ID |
|---|---|---|---|---|
| Gene | Sequences | NO: | Sequences | NO: |
| Endogenous | TCT TTC CAC CAG GCC | 1 | TGC GGG CGG ACA | 27 |
| Oct3/4 | CCC GGC TC | | TGG GGA GAT CC | |
| Total Oct3/4 | GTG AGG GCC AGG | 2 | CTG TAG GGA GGG | 28 |
| | CAG GAG CAC GAG | | CTT CGG GCA CTT | |
| Endogenous Sox2 | TAG AGC TAG ACT CCG | 3 | TTG CCT TAA ACA | 29 |
| | GGC GAT GA | | AGA CGA CGA AA | |
| Total Sox2 | GGT TAC CTC TTC CTC | 4 | TCA CAT GTG CGA | 30 |
| | CCA CTC CAG | | CAG GGG CAG | |
| Endogenous Klf4 | GCG AAC TCA CAC | 5 | TCG CTT CCT CTT | 31 |
| | AGG CGA GAA ACC | | CCT CCG ACA CA | |
| Total Klf4 | CAC CAT GGA CCC | 6 | TTA GGC TGT TCT | 32 |
| | GGG CGT GGC TGC | | TTT CCG GGG CCA | |
| | CAG AAA | | CGA | |
| Endogenous c- | TGA CCT AAC TCG AGG | 7 | AAG TTT GAG GCA | 33 |
| Myc | AGG AGC TGG AAT C | | GTT AAA ATT ATG | |
| | | | GCT GAA GC | |
| Total c-Myc | CAG AGG AGG AAC | 8 | TTA TGC ACC AGA | 34 |
| | GAG CTG AAG CGC | | GTT TCG AAG CTG | |
| | | | TTC G | |
| Gapdh | GAT GCC CCC ATG TTT | 9 | TTG CTG ACA ATC | 35 |
| | GTG AT | | TTG AGT GAG TTC | |
| | | | T | |
| Sox 17 | CCA TAG CAG AGC | 10 | GTG CGG AGA CAT | 36 |
| | TCG GGG TC | | CAG CGG AG | |
| HNF3β (Foxa2) | ACT GGA GCA GCT ACT | 11 | CCC ACA TAG GAT | 37 |
| | ACG | | GAC ATG | |
| Cytokeratin 19 | CTG CAG ATG ACT TCA | 12 | GGC CAT GAT CTC | 38 |
| | GAA CC | | ATA CTG AC | |
| Isl-1 | GTT TGT ACG GGA TCA | 13 | ATG CTG CGT TTC | 39 |
| | AAT GC | | TTG TCC TT | |
| Nestin | CTA CCA GGA GCG | 14 | TCC ACA GCC AGC | 40 |
| | CGT GGC | | TGG ACC TT | |
| Ngn3 (MATH4B) | TGG CGC CTC ATC CCT | 15 | AGT CAC CCA CTT | 41 |
| | TGG ATG | | CTG CTT CG | |
| Pax4 | ACC AGA GCT TGC ACT | 16 | CCC ATT TCA GCT | 42 |
| | GGA. CT | | TCT CTT GC | |
| Pax6 | TCA CAG CGG AGT | 17 | CCC AAG CAA AGA | 43 |
| | GAA TCA G | | TGG AAG | |
| Pdx1 (IPF-1) | CTT TCC CGT GGA TGA | 18 | GTC AAG TTC AAC | 44 |
| | AAT CC | | ATC ACT GCC | |
| Insulin 1 | TAG TGA CCA GCT ATA | 19 | CGC CAA GGT CTG | 45 |
| | ATC AGA GAC | | AAG GTC | |
| Glucagon | CAT TCA CAG GGC | 20 | CCA GCC CAA GCA | 46 |
| | ACA TTC ACC | | ATG AAT TCC | |
| Amylase | CAG GCA ATC CTG | 21 | CAC TTG CGG ATA | 47 |
| | CAG GAA CAA | | ACT GTG CCA | |
| Glut-2 | TTC GGC TAT GAC ATC | 22 | AGC TGA GGC CAG | 48 |
| | GGT GTG | | CAA TCT GAC | |
| IAPP | TGA TAT TGC TGC CTC | 23 | GGA GGA CTG GAC | 49 |
| | GGA CC | | CAA GGT TG | |
| PP | ACT AGC TCA GCA CAC | 24 | AGA CAA GAG AGG | 50 |
| | AGG AT | | CTG CAA GT | |
| Somatostatin | TCG CTG CTG CCT GAG | 25 | GCC AAG AAG TAC | 51 |
| | GAC CT | | TTG GCC AGT TC | |
| β5-tubulin | TCA CTG TGC CTG AAC | 26 | GGA ACA TAG CCG | 52 |
| | TTA CC | | TAA ACT GC | |

TABLE 2
Markers used for Immunostaining

| | Primary ab* and | Secondary ab* and | Blocking |
|---|---|---|---|
| Antigen | Dilutions | Dilutions | Buffer |
| GFP | Rabbit polyclonal, 1:100 | Donkey anti-rabbit, 1:1000 | 5% BSA |
| | (Cell Signaling) | PE (BD Pharmingen) | (Fisher Scientific) |
| Oct 3/4 | Rabbit polyclonal, 1:100 | Donkey anti-rabbit, 1:1000 | 5% BSA |
| | (Santa Crux Biotech.) | PE (BD Pharmingen) | (Fisher Scientific) |
| Sox2 | Rabbit polyclonal, 1:100 | Donkey anti-rabbit, 1:1000 | 5% BSA |
| | (Santa Cruz Biotech) | PE (BD Pharmingen) | (Fisher Scientific) |
| Nanog | Rabbit polyclonal, 1:100 | Donkey anti-rabbit, 1:1000 | 5% BSA |
| | (Santa Cruz Biotech) | PE (BD Pharmingen) | (Fisher Scientific) |
| SSEA1 | Mouse polyclonal, 1:100 | Donkey anti-mouse, 1:1000 | 5% BSA |
| | (Santa Cruz Biotech) | PE (BD Pharmingen) | (Fisher Scientific) |
| Nestin | Mouse monoclonal, 1:100 | Donkey anti-mouse, 1:1000 | 5% BSA |
| | (Millipore) | PE (BD Pharmingen) | (Fisher Scientific) |
| Insulin | Mouse monoclonal, 1:100 | Goat anti-mouse, 1:1000 | 5% BSA |
| | (Sigma) | PE (BD Pharmingen) | (Fisher Scientific) |
| Islet | Rabbit polyconal, 1:100 | Goat anti-rabbit, 1:1000 | 5% BSA |
| | (Abcam) | PE (BD Pharmingen) | (Fisher Scientific) |

*Primary and Secondary antibodies were diluted to 1% Blocking Buffer before use in immunofluorescence staining.

We claim:

1. A method of treating diabetes in a subject comprising administering β-like cells derived from induced pluripotent stem (iPS) cells to said subject, wherein said β-like cells are insulin secreting cells.

2. The method of claim 1 wherein the subject is a human subject.

3. The method of claim 1 wherein the subject has Type 1 diabetes.

4. The method of claim 1 wherein the subject has Type 2 diabetes.

5. The method of claim 1 wherein the β-like cells are administered by engrafting to liver parenchyma via intraportal vein injection.

6. The method of claim 1 wherein the iPS cells are autologous iPS cells derived from the subject.

7. The method of claim 1 wherein at least about 200,000 β-like cells are administered.

8. The method of claim 1 wherein the iPS cells are derived from fibroblasts,

9. The method of claim 1 wherein the iPS cells are derived from adult somatic cells wherein generation of multi-lineage progenitors is performed without trypsinization.

10. The method of claim 1 wherein the β-like cells are administered when the subject is responsive to insulin.

11. The method of claim 1 wherein the β-like cells are administered when the subject is resistant to insulin.

12. The method of claim 1, wherein the β-like cells secrete insulin for at least about 8 weeks.

13. The method of claim 1, wherein the β-like cells further comprise α-like cells.

14. Cells comprising β-like cells derived from induced pluripotent stem (iPS) cells, wherein said β-like cells are insulin secreting cells.

15. The β-like cells of claim 14, wherein the β-like cells are human cells.

16. The (iPS) cells of claim 14, wherein the iPS cells are derived from adult somatic cells wherein generation of multi-lineage progenitors is performed without trypsinization.

17. The cells of claim 14, wherein the iPS cells are derived from fibroblasts.

18. The cells of claim 14, wherein the β-like cells secrete insulin for at least about 8 weeks.

19. A method of generating an induced pluripotent stem cell (iPS) comprising transforming an adult somatic cell with a vector that expresses transcription factors that induce pluripotency, wherein the transcription factors comprise Oct4, Nanog, and Sox2.

20. The method of claim 19, wherein the transcription factors further comprise Lin28.

21. The method of claim 19, wherein the adult somatic cell is a human cell.

22. The method of claim 19, wherein the adult somatic cell is a fibroblast.

23. The method of claim 19, wherein the vector can not replicate in the transformed cell.

24. The method of claim 19, wherein the vector does not integrate into the cell's chromosome.

25. The method of claim 19, wherein the vector is derived from a baculovirus.

26. An induced pluripotent stem cell (iPS) produced by the method according to any one of claims 19-25.

27. A method of generating a fate-controllable stem cell comprising integrating a heterologous DNA into a chromosome of the cell by homologous recombination, wherein the heterologous DNA encodes a suicide gene comprising caspase-9, caspase-8, caspase-2, BH3 interacting domain death agonist (BID), or Herpes Simplex Virus-1 thymidine kinase.

28. The method of claim 27, wherein the stem cell is a human cell.

29. The method of claim 27, wherein the stem cell is an induced pluripotent stem cell (iPS).

30. The method of claim 27, wherein the heterologous DNA comprises an inducible promoter.

31. The method of claim 30, wherein the inducible promoter can be turned on by tetracycline or a tetracycline derivative.

32. The method of claim 27, wherein the heterologous DNA comprises a drug selection cassette.

33. A stem cell produced by e method according to any one of claims 27-32.
